(19) United States
(12) Patent Application Publication
(10) Pub. No.: US 2026/0207526 A1
(43) Pub. Date: Jul. 23, 2026

(54) LIPID NANOPARTICLES LYOPHILIZATION METHODS AND COMPOSITIONS

(71) Applicant: GLOBAL LIFE SCIENCES SOLUTIONS CANADA ULC, Vancouver (CA)

(72) Inventors: Anitha Thomas, Vancouver (CA); Srinivas Abbina, Vancouver (CA); Helena Son, Vancouver (CA); Hossein Yazdani-Ahamad-Abadi, Vancouver (CA)

(21) Appl. No.: 19/137,961

(22) PCT Filed: Dec. 11, 2023
(86) PCT No.: PCT/EP2023/085214

§ 371 Date: Jun. 11, 2025

Related U.S. Application Data
(60) Provisional application No. 18/079,482, filed on Dec. 12, 2022.

(51) Int. Cl. A61K 9/51; A61K 9/00; A61K 9/19; A61K 39/00; A61K 39/12; A61K 47/26; A61K 47/34; A61P 31/14; C12N 15/88

(52) U.S. Cl. CPC A61K 9/5123; A61K 9/0019; A61K 9/19; A61K 39/12; A61K 47/26; A61K 47/34; A61P 31/14; C12N 15/88; A61K 2039/53; A61K 2039/54; A61K 2039/55555; C12N 2770/20034

(57) ABSTRACT

The invention provides a lyophilized nucleic acid lipid nanoparticle (NALNP) comprising (a) a lipid nanoparticle comprising a nucleic acid, and (b) a lyophilization buffer comprising a sugar, a lyophilization reagent, and a pharmaceutically acceptable diluent, as well as a method of preparing same.

### CROSS-REFERENCE TO RELATED APPLICATIONS

This application claims priority under 35USC § 119 (e) of U.S. patent application Ser. No. 18/479,482 filed on Dec. 12, 2022.

### BACKGROUND OF THE INVENTION

Because of the short production times, mRNA vaccines are ideal for responding quickly to new threats like the COVID-2019 virus. Furthermore, nucleic-acid-based vaccines offer advantages over traditional vaccines in terms of safety and efficacy. Messenger RNA (“mRNA”) vaccines compare favorably against vaccines based on DNA, which need to cross the nuclear membrane in order to work and carry the risk of integration into the host genome. Messenger RNA vaccines would be subject to degradation by exonucleases and endonucleases in vivo without a delivery system, so mRNA vaccines need a carrier. Currently, lipid nanoparticles (LNPs) are among the most frequently used vectors for in vivo RNA delivery. Lipid nanoparticles or LNPs generally consist of a lipid or aqueous core surrounded by a lipid bilayer shell that is made of a combination of different lipids, each serving distinct functions.

Current mRNA vaccines must be kept frozen at low temperatures for storage. This requirement limits their distribution. Thus a suitable lyophilization protocol is still required to maintain mRNA vaccines in a stable and effective form.

### BRIEF SUMMARY OF THE INVENTION

In accordance with an embodiment, there is provided a lyophilized nucleic acid lipid nanoparticle (NALNP) including;
(a) a lipid nanoparticle comprising a nucleic acid, and
(b) a lyophilization buffer comprising a sugar, and a lyophilization reagent chosen from a sugar mimicking oligomer/polymer, an amphiphilic thermosresponsive polymer, an ethylene glycol mimicked polymer, a hydrophilic monomer and a hydrophilic polymer.

In some embodiments, the the sugar mimicking oligomer/polymer is n-octanoylsucrose, cyclodextrin, beta-cyclodextrin polymer, dextran, trehalose, carboxy terminated PEG with sorbitol core, Betadex™ sulfobutyl ether sodium, or 2-hydroxylpropyl-beta-cyclodextrin.

In other embodiments, the amphiphilic thermosresponsive polymer is poly(N-vinyl caprolactam), poly(N, N-dimethyl acrylamide), poly(N,N-diethyl acrylamide), or poly(acrylamide).

In other embodiments, the ethylene glycol mimicked polymer is 6-arm branched PEG, 5-arm branched PEG, 3-arm branched PEG, trimethyl propane ethoxylate, polyethylene glycol, Poloxamer 407, amine terminated 4-arm PEG, glycerol ethoxylate, or poly(propylene glycol).

In still other embodiments, the hydrophilic monomer or polymer is propylene glycol, glycerol, poly-propylene glycol, triglycerol, poly(vinylpyrrolidone), poly(2-ethyl-2-oxazoline), amino acids, or L-arginine.

In embodiments of the invention, the wt/wt ratio of lyophilization reagent to sugar is 1:8 or 1:4. In embodiments, the pharmaceutically acceptable diluent is selected from solutions of Tris, sodium acetate, sodium citrate, dextrose, and saline. In embodiments, the NALNP has a wt/wt ratio of nucleic acid to lyophilization reagent to sugar of 1:125:1000, 1:250:1000, 1:250:2000 or 1:500:2000. In embodiments, the sugar is sucrose. In many embodiments, the NALNP is in an anhydrous form.

In accordance with an embodiment, there is provided a method for preparing a lyophilized NALNP including
(a) mixing a nucleic acid with a lipid mix solution comprising an ionizable lipid, a structural lipid, a sterol, and a stabilizing agent to form a lipid nanoparticle;
(b) combining the lipid nanoparticle obtained in (a) with a lyophilization buffer comprising a sugar and a lyophilization reagent selected from the group consisting of polyvinylpyrrolidone, poly(N-vinyl caprolactam), 2-hydroxylpropyl-beta-cyclodextrin, N, N-dimethyl acrylamide, poly(N,N-diethyl acrylamide), poly(2-ethyl-2-oxazoline), glycerol ethoxylate, amine terminated 4-arm PEG, 6-arm branched PEG, 5-arm branched PEG, and carboxy terminated PEG with sorbitol core, and a pharmaceutically acceptable diluent; and
(c) lyophilizing the combined lipid nanoparticle and lyophilization buffer obtained in (b) to obtain an anhydrous lyophilized NALNP.

In embodiments, lyophilizing the combined nanoparticle and lyophilization buffer includes:
(a) freezing the combined lipid nanoparticle and lyophilization buffer at −40 to −90° C. for 60-400 minutes,
(b) drying the combined lipid nanoparticle and lyophilization buffer at −20 to −40° C. and 30-100 mTorr for 700-980 minutes, and
(c) drying the combined lipid nanoparticle and lyophilization buffer at 4-10° C. at 30-100 mTorr for 250-500 minutes. In embodiments, the lyophilization buffer comprises a pharmaceutically acceptable diluent.

In embodiments, the pharmaceutically acceptable diluent is Tris, sodium acetate, sodium citrate, dextrose, saline or water. In some embodiments, the lyophilization buffer has a wt/wt ratio of lyophilization reagent to sugar to pharmaceutically acceptable diluent between 1:4:40 and 1:8:80. In embodiments, the NALNP has a wt/wt ratio of nucleic acid to lyophilization reagent to sugar of 1:125:1000, 1:250:1000, 1:250:2000 or 1:500:2000.

In embodiments, the sugar is sucrose.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE SEVERAL VIEWS OF THE DRAWING

FIG. 1 is a schematic of an example lyophilization process according to the present invention;

FIG. 2 is a graph showing the size and polydispersity results for the lyophilized LNPs, reconstituted in 1× phosphate-buffered saline (PBS) after storage at room temperature (RT) for 24 hour (hr), formulated using two different saRNAs (SARS-COV-2 spike protein specific A3b or A3p antigens) in VA composition at N/P-8;

FIG. 3 is a graph showing the encapsulation efficiency results for the lyophilized LNPs, using lyophilization buffers (LB) #s 107 and 110, stored at RT for 24 hr reconstituted in 1×PBS, made using two different saRNAs (SARS-COV-2 spike protein specific A3b or A3p antigens) in VA composition at N/P-8;

FIG. 4 shows the western blot results of SARS-COV-2 spike protein expressed in vitro in HEK-293 cells that were treated with lyophilized LNPs comprising PNI 516 with LB #s 107, 110, 163, 164, 168, 178, 179, 180, 181, 182, 186 and 189, stored at 4° C. for 24 hr, reconstituted in 1×PBS, at 1 μg/mL of saRNA for 24 h;

FIG. 5 is a western blot image showing the SARS-COV-2 spike protein expression in HEK 293 cells, treated with lyophilized PNI 516 LNPs encapsulating SARS-COV-2 spike protein specific A3saRNA in VB composition (N/P-8), lyophilized and stored at −20° C. and 4° C. for one week, and then reconstituted;

FIG. 6 is showing the SARS-COV-2 spike protein expression in HEK 293 cells determined by western blot analysis. The LNPs, encompassing PNI 516 and SARS-COV-2 spike protein specific A3saRNA, were formulated in VB composition at N/P-8, lyophilized, stored at RT for one week, and reconstituted;

FIG. 7 shows the western blot analysis results for SARS-COV-2 specific spike protein expression in HEK 293 cells after treating with lyophilized LNPs at the concentration of 0.25 μg/mL of saRNA. The LNPs were formulated with PNI 516 and SARS-COV-2 spike protein specific A5 saRNA in VB composition at N/P-8. After lyophilization, the lyophilized cakes were stored for three months at RT and 4° C. and reconstituted in 1×PBS;

FIG. 8 shows a graph of SARS-COV-2 spike protein specific IgG expression in C57BL/6 mice on Day 42 following IM administration of 1 μg/mouse dose of SARS-COV-2 spike protein encoded saRNA LNPs, containing PNI 516 in VB composition (N/P-8) after lyophilization, storage at three different temperatures (−20° C., 4° C., and RT) for one week, and reconstitution;

FIG. 9 is a graph showing the human Erythropoietin (hEPO) protein expression in HEK293 cells treated with lyophilized LNPs, stored at three different temperatures (20° C., 4° C., and RT) for one week, reconstituted in 1×PBS, at a dose of 1 μg/mL;

FIG. 10A-10E are graphs showing the EPO protein expression levels in HEK293 cells treated with lyophilized and reconstituted LNPs that were stored at RT for one week, in 1×PBS at a dose of 1 μg/mL. These EPO mRNA-LNPs containing either PNI 516 (FIG. 10A), PNI 127 (FIG. 10B), dilinoleylmethyl-4-dimethylaminobutyrate (DLin-MC3-DMA) (FIG. 10C), 2-dilinoleyl-4-dimethylaminoethyl-[1,3]-dioxolane (DLin-KC2-DMA) (FIG. 10D), or butanoic acid, 4-(dimethylamino)-9-(2-octylcyclopropyl)-1-[8-(2-octylcyclopropyl) octyl]nonyl ester (BOCHD-C3-DMA) (FIG. 10E) in VB composition at N/P-8;

FIG. 11A is a graph showing the encapsulation efficiency results for the reconstituted lyophilized EPO mRNA-LNPs in 1×PBS that were stored at RT for one week and formulated with PNI 516;

FIG. 11B is a graph showing the encapsulation efficiency results for the reconstituted lyophilized EPO mRNA-LNPs in 1×PBS that were stored at RT for one week and formulated with PNI 127;

FIG. 11C is a graph showing the encapsulation efficiency results for the reconstituted lyophilized EPO mRNA-LNPs in 1×PBS that were stored at RT for one week and formulated with dilinoleylmethyl-4-dimethylaminobutyrate (DLin-MC3-DMA);

FIG. 11D is a graph showing the encapsulation efficiency results for the reconstituted lyophilized EPO mRNA-LNPs in 1×PBS that were stored at RT for one week and formulated with 2-dilinoleyl-4-dimethylaminoethyl-[1,3]-dioxolane (DLin-KC2-DMA);

FIG. 11E is a graph showing the encapsulation efficiency results for the reconstituted lyophilized EPO mRNA-LNPs in 1×PBS that were stored at RT for one week and formulated with butanoic acid, 4-(dimethylamino)-9-(2-octylcyclopropyl)-1-[8-(2-octylcyclopropyl) octyl]nonyl ester (BOCHDC3-DMA);

FIG. 12A is a graph showing the size and polydispersity results for the reconstituted lyophilized EPO mRNA-LNPs in 1×PBS after storage at RT for one week, formulated with PNI 516;

FIG. 12B is a graph showing the size and polydispersity results for the reconstituted lyophilized EPO mRNA-LNPs in 1×PBS after storage at RT for one week, formulated with PNI 127;

FIG. 12C is a graph showing the size and polydispersity results for the reconstituted lyophilized EPO mRNA-LNPs in 1×PBS after storage at RT for one week, formulated with dilinoleylmethyl-4-dimethylaminobutyrate (DLin-MC3-DMA);

FIG. 12D is a graph showing the size and polydispersity results for the reconstituted lyophilized EPO mRNA-LNPs in 1×PBS after storage at RT for one week, formulated with 2-dilinoleyl-4-dimethylaminoethyl-[1,3]-dioxolane (DLin-KC2-DMA);

FIG. 12E is a graph showing the size and polydispersity results for the reconstituted lyophilized EPO mRNA-LNPs in 1×PBS after storage at RT for one week, formulated with butanoic acid, 4-(dimethylamino)-9-(2-octylcyclopropyl)-1-[8-(2-octylcyclopropyl) octyl]nonyl ester (BOCHD-C3-DMA);

FIG. 13A is a graph showing the EPO protein expression levels in HEK293 cells treated with lyophilized and reconstituted LNPs in 1×PBS after storage at RT for one week at a dose of 1 μg/mL. The LNPs of PNI 516 and EPO mRNA-LNPs were varied with different helper lipids DSPC, DPPC, DOPE, or DOPC in VB composition (N/P-8) and lyophilized with LB #107 buffer;

FIG. 13B is a graph showing the EPO protein expression levels in HEK293 cells treated with lyophilized and reconstituted LNPs in 1×PBS after storage at RT for one week at a dose of 1 μg/mL. LNPs, of PNI 516 and EPO mRNA-LNPs with helper lipid DSPC were lyophilized using LB #110 and LB #189 buffers;

FIG. 13C is a graph showing the EPO protein expression levels in HEK293 cells treated with lyophilized and reconstituted LNPs in 1×PBS after storage at RT for one week at a dose of 1 μg/mL. LNPs, of PNI 516 and EPO mRNA-LNPs with helper lipid DPPC were lyophilized using LB #107 and LB #189 buffers;

FIG. 14 is a graph showing the Luciferase protein expression levels in HEK293 cells treated with lyophilized and reconstituted LNPs containing PNI 516 or PNI 127 in 1×PBS after storage at RT for one week at a dose of 25 ng/well;

FIG. 15 is a graph showing EPO expression level in C57BL/6 mice following i.v administration of 0.25 mg/Kg dose of PNI 516 and recombinant human EPO-encoded mRNA LNPs (VB composition, N/P-8) which were lyophilized and reconstituted in 1×PBS prior to administration after storage at three different temperatures (−20° C., 4° C., and RT) for one week prior to the treatment;

FIG. 16 is a graph showing the encapsulation efficiency results for the lyophilized LNPs, made from PNI 516 and EPO-encoded mRNA, using 3 different lyophilization buffers, after administration in mice. The lyophilized cakes were stored at three different temperatures (−20° C., 4° C., and RT) for one week and reconstituted in 1×PBS prior to the treatment;

FIG. 17 is a graph showing the size and PDI results for the lyophilized LNPs, made from PNI 516 and EPO-encoded mRNA, using 3 different lyophilization buffers, after administration in mice. The lyophilized cakes were stored at three different temperatures (−20° C., 4° C., and RT) for one week and reconstituted in 1×PBS prior to the treatment;

FIG. 18 is a photographic image of the western blot of cell lysates for HEK-293cells treated with post lyophilization LNP containing of SARS-COV-2 spike protein mRNA expressed in vitro (0.25 μg/mL of saRNA for 24 h), the LNP lyophilized with lyophilization buffer comprising PNI 516 with LB #s 3, 9, 31, 54, 98, 101, 112, 113, 119, 120, 134, 135, 136, 142, 143, 147, 148, 157, 161, 162, 163, 164, 166, 167, 168, 170, 180, 181, 182, 187, 188 and 208, LNP stored at RT for 12 days, and reconstituted in 1×PBS prior to treatment;

FIG. 19 shows a graphical representation of the EPO protein level in sera of mice treated with the lyoprotected (LB #s 54, 108, 167, 218, 241, and 260) LNPs following 2 months storage at 4° C.; and

FIG. 20 is a graphical representation of the SARS-COV-2 Spike protein specific IgG level in sera of mice treated with the dosing materials containing lyophilization buffer leads LB #s 354, 108, 164, and 167 after LNP storage of 1 month at 4° C.

### DETAILED DESCRIPTION OF THE INVENTION

The inventive lyophilized nucleic acid lipid nanoparticle (NALNP) comprises (a) a lipid nanoparticle comprising a nucleic acid, and (b) a lyophilization buffer comprising a sugar, a lyophilization reagent, and a pharmaceutically acceptable diluent.

The inventive lyophilized NALNP comprises a lipid nanoparticle comprising a nucleic acid. Lipid nanoparticles are a subgroup of lipid particles with a mean diameter of from about 15 to about 300 nm. In some embodiments, the mean particle diameter is greater than 200 nm. In some embodiments, the lipid particle has a diameter of about 300 nm or less, 250 nm or less, 200 nm or less, 150 nm or less, 100 nm or less, or 50 nm or less. In one embodiment, the lipid particle has a diameter of from about 50 to about 150 nm. Smaller particles generally exhibit increased circulatory lifetime in vivo compared to larger particles. Smaller particle shave an increased ability to reach tumor sites than larger nanoparticles. In one embodiment, the lipid particle has a diameter from about 15 to about 50 nm.

Lipid nanoparticles are generally spherical assemblies of lipids, nucleic acid, sterols, and stabilizing agents. Positive and negative charges, ratios, as well as hydrophilicity and hydrophobicity dictate the physical structure of the lipid particles in terms of size and orientation of components. The structural organization of these lipid may lead to an aqueous interior with a minimum bilayer as in liposomes or it may have a solid interior as in a solid nucleic acid lipid nanoparticle. There may be phospholipid monolayers or bilayers in single or multiple forms. Lipid particles are between 1 and 1000 μm in size.

In some embodiments, lipid nanoparticles comprise a lipid mix solution and nucleic acid. In some embodiments, lipid mix solution comprises an ionizable lipid, a structural lipid, a sterol, and a stabilizing agent. “N/P” is the ratio of moles of the amine groups of ionizable lipids to those of the phosphate groups of nucleic acid. In some embodiments, the N/P ratio is 4-12. In a preferred embodiment, the N/P ratio is 6-10. For example, in preferred embodiments, the N/P ratio is 6, 8, or 10. The nucleic acid is associated with the lipid mix composition to form a LNP in a premeditated ratio such as ionizable lipid amine (N) to nucleic acid phosphate ration (P) of N/P 4, N/P 6, N/P 8, N/P 10, N/P 12 or any other suitable N/P ratio. In some embodiments, a lipid mix solution comprises a stabilizing agent or stabilizer. Any suitable stabilizing agent or stabilizer can be used in embodiments of the present invention. In some embodiments, the stabilizing agent is chosen from polysorbates (Tweens), Brij™ S20 (polyoxyethylene (20) stearyl ether), Brij™35 (Polyoxyethylene lauryl ether, Polyethyleneglycol lauryl ether), Brij™S10 (Polyethylene glycol octadecyl ether, Polyoxyethylene (10) stearyl ether), Myrj™52 (polyoxyethylene (40) stearate), PEG-DMG, PEG-DMG 2000, Triolein, Tridecyl-D-maltoside, Tween 20, Polysorbate 80, Lipid H, TPGS1000, polyoxyethylene (4) lauryl ether, and DiD. Stabilizing agent combinations are also used in some embodiments, including polysorbate and maltoside, Alkyl polyglycosides (TBD), PEG-conjugated lipids or other polymer conjugated lipids. In some embodiments, the lipid mix solution comprises more than one stabilizing agent or stabilizers. For example, in some embodiments, the lipid mix solution comprises one or more, two or more, three or more, or four or more stabilizing agents or stabilizers.

In some embodiments, a lipid mix solution comprises an ionizable lipid. Any suitable ionizable lipid can be used in embodiments of the present invention. An ionizable lipid is a lipid that is cationic or becomes ionizable (protonated) as the pH is lowered below the pKa of the ionizable group of the lipid but is more neutral at higher pH values. At pH values below the pKa, the lipid is then able to associate with negatively charged nucleic acids (e.g., oligonucleotides). Examples of suitable ionizable lipids are found in PCT Publication Nos. WO2020252589 and WO2021000041.

In some embodiments, the ionizable lipid is DODMA (1,2-dioleyloxy-3-dimethylaminopropane), DLin-MC3-DMA (O—(Z,Z,Z,Z-heptatriaconta-6,9,26,29-tetraen-19-yl)-4-(N,N-dimethylamino)), DLin-KC2-DMA (2-dilinoleyl-4-dimethylaminoethyl-[1,3]-dioxolane), butanoic acid, BOCHD-C3-DMA (4-(dimethylamino)-9-(2-octylcyclopropyl)-1-[8-(2 octylcyclopropyl) octyl]nonyl ester), or C12-200. In some preferred embodiments, the ionizable lipid is (Z)-3-(2-((1,17-bis(2-octylcyclopropyl) heptadecan-9-yl)oxy)-2-oxoethyl)-2-(pent-2-en-1-yl)cyclopentyl 4-(dimethylamino)butanoate (referred to as PNI 516) (WO 2020/252589) or (2R,3S,4R)-2-(((1,4-dimethylpiperidine-4-carbonyl)oxy)methyl)tetrahydrofuran-3,4-diyl(9E,9′E,12E,12′E)-bis(octadeca-9,12-dienoate) (referred to as PNI 127) (WO 2021/000041).

In some embodiments a lipid mix solution comprises a structural lipid. A structural lipid can also be known as a helper lipid or neutral lipid. Any suitable structural lipid can be used in the embodiments of the present invention. Suitable structural lipids support the formation of particles during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides.

Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), 1-stearoyl-2-oleoyl-sn-glycero-3-phosphocholine (SOPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE). In one preferred embodiment, the structural lipid is distearoylphosphatidylcholine (DSPC).

In another embodiment, the structural lipid is any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

In some embodiments, the lipid mix solution comprises a sterol. Any suitable sterol can be used. In some embodiments, the sterol is cholesterol, beta-sitosterol, 20-alpha-hydroxysterol, or phytosterol. In a preferred embodiment, the sterol is cholesterol.

The lipid mix can comprise any suitable combination of ionizable lipid, structural lipid, sterol, and stabilizing agent. In some embodiments, the lipid mix comprises 47.5 mol % ionizable lipid, 12.5 mol % structural lipid, 38.5 mol % sterol, and 1.5 mol % stabilizing agent. In a preferred embodiment, the lipid mix comprises 47.5 mol % IL, 13.5 mol % DOPE, 38.5 mol % cholesterol, and 1.5 mol % PEG-DMG. In another preferred embodiment, the lipid mix comprises 47.5 mol % IL, 122.5 mol % DSPC, 38.5 mol % cholesterol, and 1.5 mol % PEG-DMG. In other embodiments, the lipid mix comprises 40 mol % ionizable lipid, 20 mol % structural lipid, 37.5 mol % sterol, and 2.5 mol % stabilizing agent. For example, in a preferred embodiment, the lipid mix comprises 40 mol % ionizable lipid, 30 mol % DSPC, 37.5 mol % cholesterol, and 2.5 mol % BRIJ™ S10.

The lipid nanoparticle comprises a nucleic acid. Any suitable nucleic acid can be used in the lipid nanoparticle. The nucleic acid is a substance intended to have a direct effect in the diagnosis, cure, mitigation, treatment or prevention of disease, or to have direct effect in restoring, correcting or modifying physiological functions, or to act as a research reagent. In some embodiments, the nucleic acid is a siRNA, miRNA, a self-amplifying RNA (SAM or saRNA), a self-replicating DNA, an LNA, a DNA, replicon, an mRNA, a guide RNA, a transposon, or a single gene. In some embodiments, the nucleic acid is referred to as a nucleic acid therapeutic or NAT.

The inventive lyophilized NALNP comprises a lyophilization buffer (sometimes referred to as lyo buffer) comprising a sugar, a lyophilization reagent, and a pharmaceutically acceptable diluent.

The inventive lyophilization buffer comprises a sugar. Any suitable sugar can be used. In some embodiments, the sugar is chosen from sucrose, mannose, mannitol, sorbitol, raffinose, fructose, glucose, lactose, maltose, maltodextrin, trehalose, inulin, and dextran. In a preferred embodiment, the sugar is sucrose. In some embodiments, the lyophilization buffer comprises more than one type of sugar. In some embodiments, the lyophilization buffer comprises one or more, two or more, or three or more types of sugars.

The inventive lyophilization buffer comprises a lyophilization reagent. In some embodiments, the lyophilization reagent is chosen from polyvinyl alcohol, a sugar mimicking oligomer/polymer, an amphiphilic thermoresponsive polymer, an ethylene glycol mimicked polymer, or hydrophilic monomer or polymer. In some embodiments, the sugar mimicking oligomer/polymer is n-octanoylsucrose, cyclodextrin, beta-cyclodextrin, dextran, trehalose, carboxy terminated PEG with sorbitol core, Betadex™ sulfobutyl ether sodium, or 2-hydroxylpropyl-beta-cyclodextrin. In some embodiments, the amphiphilic thermosresponsive polymer is poly(N-vinyl caprolactam), poly(N, N-dimethyl acrylamide), poly(N,N-diethyl acrylamide), or poly(acrylamide). In some embodiments, the ethylene glycol mimicked polymer is 6-arm branched PEG, 5-arm branched PEG, 3-arm branched PEG, trimethyl propane ethoxylate, polyethylene glycol, Pluronic™ (F-127), amine terminated 4-arm PEG, glycerol ethoxylate, or poly(propylene glycol). In some embodiments, the hydrophilic monomer or polymer is propylene glycol, glycerol, poly-propylene glycol, triglycerol, poly(vinylpyrrolidone), poly(2-ethyl-2-oxazoline), amino acids, or L-arginine.

In a preferred embodiment, the lyophilization reagent is polyvinylpyrrolidone. In another preferred embodiment, the lyophilization reagent is 2-hydroxylpropyl-beta-cyclodextrin. The inventive lyophilization buffer comprises a pharmaceutically acceptable diluent. Any suitable pharmaceutically acceptable diluent can be used. In some embodiments, the pharmaceutically acceptable diluent is chosen from solutions of Tris, sodium acetate, dextrose, 5% dextrose, saline, PBS, lactated Ringer's solution, 5% human serum albumin, and water. In a preferred embodiment, the pharmaceutically acceptable diluent is Tris buffer. In some embodiments, the lyophilization buffer comprises more than one pharmaceutically acceptable diluent. For example, in some embodiments, the lyophilization buffer comprises one or more, two or more, or three or more types of pharmaceutically acceptable diluents. In a preferred embodiment, the lyophilization buffer comprises Tris buffer and PBS. In some embodiments, the pharmaceutically acceptable diluent has a salt concentration of 0-70 mg/mL. For example, in some embodiments, the pharmaceutically acceptable diluent has a salt concentration of 0 mg/mL, 5 mg/mL, 10 mg/mL 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, or 70 mg/mL, or a salt concentration between any two of the aforementioned values.

In some embodiments, the lyophilization buffer comprises a base composition buffer (also referred to as BC). In some embodiments, the base composition buffer comprises a sugar and a pharmaceutically acceptable diluent as described herein. For example, in a preferred embodiment, the base composition buffer comprise 10% (W/V) of sucrose dissolved in 20 mM Tris buffer and 1×PBS (10 mg/mL). In some embodiments, the lyophilization buffer comprises a base composition buffer and lyophilization reagent. In a preferred embodiment, the lyophilization buffer comprises lyophilization reagent in an amount of 1-5% W/V. For example, in some embodiments, the lyophilization buffer comprises lyophilization reagent in an amount of 1% W/V, 1.5% W/V, 2% W/V, 2.5% W/V, 3% W/V, 3.5% W/V, 4% W/V, 4.5% W/V, 5% W/V, or a % W/V between any two of the aforementioned values.

In some embodiments, the lyophilization buffer has a wt/wt ratio of lyophilization reagent to sugar between 1:1 and 1:20. For example, in some embodiments, the lyophilization buffer has a wt/wt ratio of lyophilization reagent to sugar of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20, or a wt/wt ratio of lyophilization reagent to sugar between any two of the aforementioned values. In a preferred embodiment, the lyophilization buffer has a wt/wt ratio of lyophilization reagent to sugar between 1:4 and 1:10. In another embodiment, the wt/wt ratio of lyophilization reagent to sugar is 1:8.

In some embodiments, the lyophilization buffer has a wt/wt ratio of lyophilization reagent to sugar to pharmaceutically acceptable diluent between 1:1:40 and 1:10:80. For example, in some embodiments the wt/wt ratio of lyophilization reagent to sugar to pharmaceutically acceptable diluent is 1:1:40, 1:2:40, 1:3:40, 1:4:40, 1:5:40, 1:6:40, 1:7:40, 1:8:40, 1:9:40, 1:10:40, or a range defined by any two of the foregoing values. In other embodiments, the wt/wt ratio of lyophilization reagent to sugar to pharmaceutically acceptable diluent is 1:1:80, 1:2:80, 1:3:80, 1:4:80, 1:5:80, 1:6:80, 1:7:80, 1:8:80, 1:9:80, 1:10:80, or a range defined by any two of the foregoing values. In other embodiments, the wt/wt ratio of lyophilization reagent to sugar to pharmaceutically acceptable diluent is 1:4:40, 1:4:45, 1:4:50, 1:4:55, 1:4:60, 1:4:65, 1:4:70, 1:4:75, 1:4:80, or a range defined by any two of the foregoing values.

In other embodiments, the wt/wt ratio of lyophilization reagent to sugar to pharmaceutically acceptable diluent is 1:8:40, 1:8:45, 1:8:50, 1:8:55, 1:8:60, 1:8:65, 1:8:70, 1:8:75, 1:8:80, or a range defined by any two of the foregoing values. In a preferred embodiment, the lyophilization buffer has a wt/wt ratio of lyophilization reagent to sugar to pharmaceutically acceptable diluent between 1:4:40 and 1:8:80.

The term “diluent” in this application refers to the liquid or lyophilized form. Thus in a lyophilized form, “diluent” is the dehydrated residue of the diluent used in the lyophilization process.

In some embodiments, the NALNP has a wt/wt ratio of nucleic acid to lyophilization reagent to sugar of 1:125:1000, 1:250:1000, 1:250:2000 or 1:500:2000.

In some embodiments, the NALNP is in an anhydrous form. The NALNP is in an anhydrous form after it is lyophilized as described herein. In some embodiments, the NALNP is in an anhydrous form consists of a lyophilized or lyo cake. In some embodiments, the NALNP is in a reconstituted form. In a reconstituted form, a lyophilized NALNP has had a pharmaceutically acceptable diluent added to the lyophilized NALNP as described herein.

The inventive method for preparing a lyophilized NALNP comprises (a) mixing a nucleic acid with a lipid mix solution comprising an ionizable lipid, a structural lipid, sterol, and a stabilizing agent to form a lipid nanoparticle; (b) combining the lipid nanoparticle obtained in (a) with a lyophilization buffer; and (c) lyophilizing the combined lipid nanoparticle and lyophilization buffer obtained in (b) to obtain an anhydrous lyophilized NALNP. The inventive method for preparing a lyophilized NALNP comprises mixing a nucleic acid with a lipid mix solution comprising an ionizable lipid, a structural lipid, sterol, and a stabilizing agent to form a lipid nanoparticle. The nucleic acid can be any suitable nucleic acid according to the embodiments of the present invention. The lipid mix solution comprising an ionizable lipid, a structural lipid, sterol, and a stabilizing agent can be any suitable lipid mix according to the embodiments of the present invention. The lipid nanoparticle can be any suitable lipid nanoparticle according to the embodiments of the present invention.

Any suitable method of mixing can be used to form the lipid nanoparticle. The lipid nanoparticles according to embodiments of the invention can be prepared by standard T-tube mixing techniques, turbulent mixing, titration mixing, agitation promoting orders self-assembly, or passive mixing of all the elements with self-assembly of elements into nanoparticles. A variety of methods have been developed to formulate lipid nanoparticles containing genetic drugs. Suitable methods are disclosed in U.S. Pat. Nos. 5,753,613 and 6,734,171, by way of example. These methods include mixing preformed lipid particles with nucleic acids in the presence of ethanol or mixing lipid dissolved in ethanol with an aqueous media containing nucleic acid and result in lipid nanoparticles with nucleic acid encapsulation efficiencies of 65-99%. Both of these methods rely on the presence of ionizable lipid to achieve encapsulation of nucleic acid and a stabilizing agent to inhibit aggregation and the formation of large structures.

Automatic micro-mixing instruments such as the NanoAssemblr® instruments (Precision NanoSystems Inc, Vancouver, Canada) enable the rapid and controlled manufacture of nanomedicines (liposomes, lipid nanoparticles, and polymeric nanoparticles).

NanoAssemblr® instruments accomplish controlled molecular self-assembly of nanoparticles via microfluidic mixing cartridges that allow millisecond mixing of nanoparticle components at the nanoliter, microlitre, or larger scale with customization or parallelization. Rapid mixing on a small scale allows reproducible control over particle synthesis and quality that is not possible in larger instruments.

Preferred methods incorporate instruments such as the microfluidic mixing devices such as the NanoAssemblr® Spark™, Ignite™, Benchtop™ and NanoAssemblr® Blaze™ in order to achieve nearly 100% of the nucleic acid used in the formation process is encapsulated in the particles in one step. In one embodiment, the lipid particles are prepared by a process by which from about 90 to about 100% of the nucleic acid used in the formation process is encapsulated in the particles.

U.S. Pat. Nos. 9,758,795 and 9,943,846, describe methods of using small volume mixing technology and novel formulations derived thereby. U.S. Pat. No. 10,159,652 describes more advanced methods of using small volume mixing technology and products to formulate different materials. U.S. Pat. No. 9,943,846 discloses microfluidic mixers with different paths and wells to elements to be mixed PCT Publication WO2017117647 discloses microfluidic mixers with disposable sterile paths. U.S. Pat. No. 10,076,730 discloses bifurcating toroidal micromixing geometries and their application to micromixing. PCT Publication No. WO2018006166 discloses a programmable automated micromixer and mixing chips therefore. Mixing cartridges having microchannels and mixing geometries for mixer instruments are available from, for example, Precision NanoSystems Inc.

In embodiments of the invention, devices for biological microfluidic mixing are used to prepare the lipid particles according to embodiments of the invention. The devices include a first and second stream of reagents, which feed into the microfluidic mixer, and lipid particles are collected from the outlet, or emerge into a sterile environment.

The first stream includes a therapeutic agent in a first solvent. Suitable first solvents include solvents in which the therapeutic agents are soluble and that are miscible with the second solvent. Suitable first solvents include aqueous buffers. Representative first solvents include citrate and acetate buffers or other low pH buffers.

The second stream includes lipid mix materials in a second solvent. Suitable second solvents include solvents in which the ionizable lipids according to embodiments of the invention are soluble, and that are miscible with the first solvent. Suitable second solvents include 1,4-dioxane, tetrahydrofuran, acetone, acetonitrile, dimethyl sulfoxide, dimethylformamide, acids, and alcohols. Representative second solvents include aqueous ethanol 90%, or anhydrous ethanol.

In one embodiment of the invention, a suitable device includes one or more microchannels (i.e., a channel having its greatest dimension less than 1 millimeter). In one example, the microchannel has a diameter from about 20 to about 300 μm. In examples, at least one region of the microchannel has a principal flow direction and one or more surfaces having at least one groove or protrusion defined therein, the groove or protrusion having an orientation that forms an angle with the principal direction (e.g., a staggered herringbone mixer), as described in U.S. Pat. No. 9,943,846, or a bifurcating toroidal flow as described in U.S. Pat. No. 10,076,730. To achieve maximal mixing rates, it is advantageous to avoid undue fluidic resistance prior to the mixing region. Thus, one example of a device has non-microfluidic channels having dimensions greater than 1000 μm, to deliver the fluids to a single mixing channel.

Less automated mixing methods and instruments such as those disclosed in Zhang, S-h et al., Chemical Eng. J. 144 (2): 324-328 (2008), and U.S. Published Patent Application US 20040262223, and Jeffs, L B et al., Pharm. Resch., 22 (3): 362-372 (2005)), are also useful in creating lipid particle compositions of the invention.

The inventive method for preparing a lyophilized NALNP comprises combining a lipid nanoparticle with a lyophilization buffer. Any suitable lyophilization buffer according to embodiments of the present invention can be used. Any suitable method for combining the lipid nanoparticle with the lyophilization buffer can be used. For example, in some embodiments, the lyophilization buffer and lipid nanoparticle are mixed. In a preferred embodiment, the lyophilization buffer and lipid nanoparticle are mixed using a pipette.

The inventive method for preparing a lyophilized NALNP comprises lyophilizing the combined lipid nanoparticle and lyophilization buffer to obtain an anhydrous lyophilized NALNP. In some embodiments, lyophilizing the combined nanoparticle and lyophilization buffer comprises: (a) freezing the combined lipid nanoparticle and lyophilization buffer at −40 to −90° C. for 60-400 minutes, (b) drying the combined lipid nanoparticle and lyophilization buffer at −20 to −40° C. and 30-100 mTorr for 700-980 minutes, and (c) drying the combined lipid nanoparticle and lyophilization buffer at 4-10° C. at 30-100 mTorr for 250-500 minutes.

The lyophilizing of the combined nanoparticle and lyophilization buffer can include freezing the combined lipid nanoparticle and lyophilization buffer. Any suitable method of freezing the lipid nanoparticle and lyophilization buffer can be used. For example, the combined lipid nanoparticle and lyophilization buffer can be directly placed in a sub-zero freezer, frozen in liquid nitrogen (such as by dipping for a suitable period of time, such as about 30 s), or placed in freezing containers with controlled freezing followed by placing in the freezer. The combined nanoparticle and lyophilization buffer can be frozen at any suitable temperature. In some embodiments, the combined lipid nanoparticle and lyophilization buffer are frozen between −40 and −90° C. For example, in some embodiments, the combined lipid nanoparticle and lyophilization buffer are frozen at −40° C., −45° C., −50° C., −55° C., −60° C., −65° C., −70° C., −75° C., −80° C., −85° C., −90° C., or within a range defined by any two of the foregoing values.

In a preferred embodiment, the combined lipid nanoparticle and lyophilization buffer are frozen at −60° C. or −80° C. The combined nanoparticle and lyophilization buffer can be frozen for any suitable length of time. In some embodiments, the combined nanoparticle and lyophilization buffer are frozen for 60-400 minutes. For example, in some embodiments, the combined lipid nanoparticle and lyophilization buffer are frozen for 60 minutes, 75 minutes, 100 minutes, 125 minutes, 150 minutes, 175 minutes, 200 minutes, 225 minutes, 250 minutes, 275 minutes, 300 minutes, 325 minutes, 350 minutes, 375 minutes, 400 minutes, or a length of time within a range defined by any two of the foregoing values.

The lyophilizing of the combined nanoparticle and lyophilization buffer can include drying or desiccation of the combined lipid nanoparticle and lyophilization buffer. In some embodiment, the combined nanoparticle and lyophilization buffer is dried one time. In some embodiments, the combined nanoparticle and lyophilization buffer is dried more than one time. For example, the combined nanoparticle and lyophilization buffer can be dried one time, two times, three times, four times, or five times. Any suitable method of drying the combined lipid nanoparticle and lyophilization buffer can be used.

In some embodiments, the combined lipid nanoparticle and lyophilization buffer are dried a first time at −20 to −40° C. and 30-100 mTorr for 700-980 minutes. The combined lipid nanoparticle and lyophilization buffer can be dried at any suitable temperature. In some embodiments, the combined lipid nanoparticle and lyophilization buffer can be dried at a temperature of −20 to −40° C. For example, the combined lipid nanoparticle and lyophilization buffer can be dried at a temperature of −20° C., −22° C., −24° C., −26° C., −28° C., −30° C., −32° C., −34° C., −36° C., −38° C., −40° C., or within a range of any two of the foregoing values. In a preferred embodiment, the combined lipid nanoparticle and lyophilization buffer is dried at a temperature of −40° C. The combined lipid nanoparticle and lyophilization buffer can be dried at any suitable pressure. In some embodiments, the combined lipid nanoparticle and lyophilization buffer are dried at a pressure of 30-100 mTorr. For example, in some embodiments, the combined lipid nanoparticle and lyophilization buffer are dried at a pressure of 30 mTorr, 35 mTorr, 40 mTorr, 45 mTorr, 50 mTorr, 55 mTorr, 60 mTorr, 65 mTorr, 70 mTorr, 75 mTorr, 80 mTorr, 85 mTorr, 90 mTorr, 95 mTorr, 100 mTorr, or within a range of any two of the foregoing values. In a preferred embodiment, the combined lipid nanoparticle and lyophilization buffer is dried at a pressure of 60 mTorr. The combined lipid nanoparticle and lyophilization buffer can be dried for any suitable period of time. In some embodiments, the combined lipid nanoparticle and lyophilization buffer is dried for 700-980 minutes. For example, in some embodiments the combined lipid nanoparticle and lyophilization buffer is dried for 700 minutes, 720 minutes, 740 minutes, 760 minutes, 780 minutes, 800 minutes, 820 minutes, 840 minutes, 860 minutes, 880 minutes, 900 minutes, 920 minutes, 940 minutes, 960 minutes, 980 minutes, or within a range of any two of the foregoing values. In a preferred embodiment, the combined lipid nanoparticle and lyophilization buffer is dried for 840 minutes.

In some embodiments, the combined lipid nanoparticle and lyophilization buffer are dried a second time. In some embodiments, the combined lipid nanoparticle and lyophilization buffer are dried a second time at 4-10° C. at 30-100 mTorr for 250-500 minutes. The combined lipid nanoparticle and lyophilization buffer can be dried a second time at any suitable temperature. In some embodiments, the combined lipid nanoparticle and lyophilization buffer can be dried at a temperature of 4 to 10° C. For example, the combined lipid nanoparticle and lyophilization buffer can be dried at a temperature of 4° C., 4.5° C., 5° C., 5.5° C., 6° C., 6.5° C., 7° C., 7.5° C., 8° C., 8.5° C., 9° C., 9.5° C., 10° C., or within a range of any two of the foregoing values. In a preferred embodiment, the combined lipid nanoparticle and lyophilization buffer is dried at a temperature of 10° C. The combined lipid nanoparticle and lyophilization buffer can be dried at any suitable pressure. In some embodiments, the combined lipid nanoparticle and lyophilization buffer are dried at a pressure of 30-100 mTorr. For example, in some embodiments, the combined lipid nanoparticle and lyophilization buffer are dried at a pressure of 30 mTorr, 35 mTorr, 40 mTorr, 45 mTorr, 50 mTorr, 55 mTorr, 60 mTorr, 65 mTorr, 70 mTorr, 75 mTorr, 80 mTorr, 85 mTorr, 90 mTorr, 95 mTorr, 100 mTorr, or within a range of any two of the foregoing values. In a preferred embodiment, the combined lipid nanoparticle and lyophilization buffer is dried at a pressure of 60 mTorr. The combined lipid nanoparticle and lyophilization buffer can be dried for any suitable period of time. In some embodiments, the combined lipid nanoparticle and lyophilization buffer is dried for 250-500 minutes. For example, in some embodiments the combined lipid nanoparticle and lyophilization buffer is dried for 250 minutes, 260 minutes, 280 minutes, 300 minutes, 320 minutes, 340 minutes, 360 minutes, 380 minutes, 400 minutes, 410 minutes, 440 minutes, 460 minutes, 480 minutes, 500 minutes, or within a range of any two of the foregoing values.

In a preferred embodiment, the combined lipid nanoparticle and lyophilization buffer is dried for 320 minutes.

In some embodiments, the method further comprises reconstituting the anhydrous lyophilized NALNP. Any suitable method can be used to reconstitute the anhydrous lyophilized NALNP. In some embodiments, the anhydrous lyophilized NALNP is combined with a solution until the resulting solution is visibly homogenous. In some embodiments, the anhydrous lyophilized NALNP is combined with a solution for at least 30 minutes. For example, in some embodiments the anhydrous lyophilized NALNP is combined with a solution for at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, or within a range of any two of the foregoing values. In some embodiments, the anhydrous lyophilized NALNP is combined with a solution at a temperature between 0° C. and 10° C. For example, in some embodiments the anhydrous lyophilized NALNP is combined with a solution at a temperature of 0° C., 1° C., 2° C., 3° C., 4° C., 5° C., 6° C., 7° C., 8° C., 9° C., or 10° C., or within a range of any two of the foregoing values. In a preferred embodiment, the anhydrous lyophilized NALNP is combined with as solution at a temperature of 4° C. In some embodiments, the solution is a pharmaceutically acceptable diluent. In some embodiments, the pharmaceutically acceptable diluent is Tris, sodium acetate, dextrose, saline or water.

### EMBODIMENTS

1. A lyophilized nucleic acid lipid nanoparticle (NALNP) comprising
(a) a lipid nanoparticle comprising a nucleic acid, and
(b) a lyophilization buffer comprising a sugar, and a lyophilization reagent chosen from is a sugar mimicking oligomer/polymer, an amphiphilic thermosresponsive polymer, an ethylene glycol mimicked polymer, a hydrophilic monomer and a hydrophilic polymer.
2. The lyophilization buffer of embodiment 1, wherein the sugar mimicking oligomer/polymer is n-octanoylsucrose, cyclodextrin, beta-cyclodextrin polymer, dextran, trehalose, carboxy terminated PEG with sorbitol core, Betadex™ sulfobutyl ether sodium, or 2-hydroxylpropyl-beta-cyclodextrin.
3. The lyophilization buffer of embodiment 1, wherein the amphiphilic thermosresponsive polymer is poly(N-vinyl caprolactam), poly(N, N-dimethyl acrylamide), poly(N,N-diethyl acrylamide), or poly(acrylamide).
4. The lyophilization buffer of embodiment 1, wherein the ethylene glycol mimicked polymer is 6-arm branched PEG, 5-arm branched PEG, 3-arm branched PEG, trimethyl propane ethoxylate, polyethylene glycol, Poloxamer 407, amine terminated 4-arm PEG, glycerol ethoxylate, or poly(propylene glycol).
5. The lyophilization buffer of embodiment 1 wherein the hydrophilic monomer or polymer is propylene glycol, glycerol, poly-propylene glycol, triglycerol, poly(vinylpyrrolidone), poly(2-ethyl-2-oxazoline), amino acids, or L-arginine.
6. The NALNP of embodiment 2, wherein the wt/wt ratio of lyophilization reagent to sugar is 1:8.
7. The NALNP of embodiment 2, wherein the wt/wt ratio of lyophilization reagent to sugar is 1:5
8. The NALNP of embodiment 1, further comprising a pharmaceutically acceptable diluent selected from solutions of Tris, sodium acetate, sodium citrate, dextrose, and saline.
9. The NALNP of embodiments 1-8, wherein the NALNP has a wt/wt ratio of nucleic acid to lyophilization reagent to sugar of 1:125:1000, 1:250:1000, 1:250:2000 or 1:500:2000.
10. The NALNP of embodiment 1, wherein the sugar is sucrose.
11. The NALNP of any one of embodiments 1-8, wherein the NALNP is in an anhydrous form.
12. A method for preparing a lyophilized NALNP comprising
(a) mixing a nucleic acid with a lipid mix solution comprising an ionizable lipid, a structural lipid, a sterol, and a stabilizing agent to form a lipid nanoparticle;
(b) combining the lipid nanoparticle obtained in (a) with a lyophilization buffer comprising a sugar and a lyophilization reagent selected from the group consisting of polyvinylpyrrolidone, poly(N-vinyl caprolactam), 2-hydroxylpropyl-beta-cyclodextrin, N, N-dimethyl acrylamide, poly(N,N-diethyl acrylamide), poly(2-ethyl-2-oxazoline), glycerol ethoxylate, amine terminated 4-arm PEG, 6-arm branched PEG, 5-arm branched PEG, and carboxy terminated PEG with sorbitol core, and a pharmaceutically acceptable diluent; and
(c) lyophilizing the combined lipid nanoparticle and lyophilization buffer obtained in (b) to obtain an anhydrous lyophilized NALNP.
13. The method of embodiment 12, wherein lyophilizing the combined nanoparticle and lyophilization buffer comprises:
(a) freezing the combined lipid nanoparticle and lyophilization buffer at −40 to −90° C. for 60-400 minutes,
(b) drying the combined lipid nanoparticle and lyophilization buffer at −20 to −40° C. and 30-100 mTorr for 700-980 minutes, and
(c) drying the combined lipid nanoparticle and lyophilization buffer at 4-10° C. at 30-100 mTorr for 250-500 minutes.
14. The method of any of embodiments 12, wherein the lyophilization buffer comprises a pharmaceutically acceptable diluent.
15. The method of embodiment 14, wherein the pharmaceutically acceptable diluent is Tris, sodium acetate, sodium citrate, dextrose, saline or water.
16. The method of any of embodiments 10-15, wherein the lyophilization buffer has a wt/wt ratio of lyophilization reagent to sugar to pharmaceutically acceptable diluent between 1:4:40 and 1:8:80.
17. The method of any of embodiments 10-15, wherein the NALNP has a wt/wt ratio of nucleic acid to lyophilization reagent to sugar of 1:125:1000, 1:250:1000, 1:250:2000 or 1:500:2000.
18. The method of any of embodiments 10-15, wherein the sugar is sucrose. The following examples further illustrate the invention but, of course, should not be construed as in any way limiting its scope.

All solvents and reagents were commercial products and used as such unless noted otherwise.

Temperatures are given in degrees Celsius. The following abbreviations are used with respect to the Examples:
hEPO-mRNA: Human erythropoietin protein mRNA
FLuc-mRNA: Firefly luciferase protein mRNA
eGFP-mRNA: Enhanced green fluorescent protein mRNA
LB: Lyophilization buffer (lyo buffer)
saRNA: Self-amplified mRNA
eGFP: A basic (constitutively fluorescent) green fluorescent protein derived from Aequorea victoria
hEPO: Human erythropoietin
h: Hour(s)
HPLC: High performance liquid chromatography
MFI: Median Fluorescence Intensity
min: Minute(s)
mL: Milliliter(s)
mmol: Millimole(s)
μL: Micro liters
PBS: Phosphate buffered saline
wt: Weight® C. or Deg C: Degrees Celsius
IL: Ionizable lipid
MC3: DLin-MC3-DMA
Tween80: Polysorbate 80
BRIJ™ L4: Polyoxyethylene (4) lauryl ether
BRIJ™ S10: Polyoxyethylene (10) stearyl ether
BRIJ™ S20: Polyoxyethylene (20) stearyl ether
BRIJ™ S35: Polyoxyethylene (23) lauryl ether
TPGS 1000: D-α-Tocopherol polyethylene glycol 1000 succinate
VA composition (VA): A lipid mix comprising 47.5 mol % IL, 12.5 mol % DOPE, 38.5 mol % cholesterol, and 1.5 mol % PEG-DMG.
VB composition (VB): A lipid mix comprising 47.5 mol % IL, 12.5 mol % DSPC, 38.5 mol % cholesterol, and 1.5 mol % PEG-DMG.
CT10 composition: A lipid mix comprising 40 mol % ionizable lipid/20 mol % DSPC/37.5 mol % Chol/2.5 mol % BRIJ™ S10.
PNI 516: (Z)-3-(2-((1,17-bis(2-octylcyclopropyl) heptadecan-9-yl)oxy)-2-oxoethyl)-2-(pent-2-en-1-yl)cyclopentyl 4-(dimethylamino)butanoate (WO 2020/252589).
PNI 127: (2R,3S,4R)-2-(((1,4-dimethylpiperidine-4-carbonyl)oxy)methyl)tetrahydrofuran-3,4-diyl (9E,9′E,12E,12′E)-bis(octadeca-9,12-dienoate) (WO 2021/000041).
DLin-MC3-DMA or MC3: dilinoleylmethyl-4-dimethylaminobutyrate.
DLin-KC2-DMA or KC2: 2-dilinoleyl-4-dimethylaminoethyl-[1,3]-dioxolane butanoic acid, BOCHD-C3-DMA: 4-(dimethylamino)-9-(2-octylcyclopropyl)-1-[8-(2 octylcyclopropyl) octyl]nonyl ester.
V101: A cloning vector template for gene of interest (GOI) encoded self-amplifying replicon DNA or RNA synthesis: Parent VEEV TC83 Replicon with subgenomic promoter containing a multiple cloning site to insert any GOIs (U.S. Pat. No. 7,425,337 by Geall et al).
A3, A5: SARS-Cov-2 spike protein encoded saRNA using V101 vector (U.S. Pat. No. 7,425,337).
Base Composition buffer (BC): 10% (W/V) of sucrose dissolved in 20 mM Tris buffer and 1×PBS (10 mg/mL).
Lyo reagent or lyophilization reagent: a component used in the lyophilization buffer (LB) 5 composition.
Lyophilization buffer or lyo buffer: A combination of lyo reagent (1-5% Weight/Volume, described in Table 1) in Base Composition (BC) buffer is defined as lyo buffer or lyophilization buffer (LB).

TABLE 1
Reagents and Sources

| | Molecular | | |
|---|---|---|---|
| Reagent | weight (Da) | Supplier | Catalogue # |
| 2-Hydroxylpropyl- | 1400 | Sigma | C0926-5G |
| beta-cyclodextrin | | | |
| Polyvinylpyrrolidone | 700, 1000, | Sigma | 360627-25G |
| | 40000 | | |
| Poly(N-vinyl caprolactam) | 1300, 1800 | Polymer | P4364A-NVCL |
| | | Source | |
| Poly(vinyl alcohol) | 9000 | Sigma | 360627 |
| (80% hydrolyzed) | | | |
| Poly(propylene glycol) | 400, 1000, | Sigma | 81350 |
| | 2000 | | |
| Poly (N,N- | 19500 | Polymer | P18100-DEAMD |
| diethyl acrylamide) | | Source | |
| Poly(2-ethyl-2-oxazoline) | 6000 | Polymer | P40161-EtOXZ- |
| | | Source | CMe2Br |
| Amine terminated | 2000 | Sigma | JKA7032-1G |
| 4-arm PEG | | | |
| Glycerol ethoxylate | 1000 | Sigma | 441864-250ML |
| Betadex™ sulfobutyl | 1451 | Sigma | Y0001998 |
| ether sodium | | | |
| 6-Arm branched PEG | 5200 | Polymer | P9587-6EOOH |
| | | Source | |
| 5-Arm branched PEG | 6000 | Polymer | P9625-5EOOH |
| | | Source | |
| 3-Arm branched PEG | 11500 | Polymer | P2229-3EOOH |
| | | Source | |
| Carboxy terminated PEG | 10000 | Polymer | P3053-6EOCOOH |
| with sorbitol core | | Source | |
| Poly(N, N- | 31000 | Polymer | NA |
| dimethyl acrylamide) | | Source | |
| Poly(acrylamide) | 24500 | Polymer | NA |
| | | Source | |
| Propylene glycol | 76 | Sigma | W294004-1KG-K |
| L-arginine | 174 | Sigma | A5006-100G |
| Glycerol | 92 | Sigma | G5516-100ML |
| Trehalose | 342 | Thermo | 182551000 |
| | | Fisher | |
| Polyethylene glycol | 400 | Sigma | 8074850050 |
| Pluronic™ (F-127) | 12500 | Sigma | P2443-250G |
| Dextran | 1500 | Sigma | 31394-25G |
| Triglycerol | 240 | Sigma | 17782-50ML |
| Beta-cyclodextrin Polymer | 2000 | Sigma | C2485-1G |
| n-Octanoylsucrose | 468 | Sigma | 494466-5GM |
| Trimethyl | 450 | Sigma | 409774-250ML |
| propane ethoxylate | | | |
| Tris ( | 121.14 | Sigma- | 252859-100G |
| Tris(hydroxymethyl) | | Aldrich | |
| aminomethane) | | | |

### Example 1

This Example demonstrates a nucleic acid therapeutic (NAT) preparation used in the Examples of the present application. Messenger RNA, saRNA, or DNA plasmid nucleic acid 5 therapeutic (NAT) as described below, was diluted using sodium acetate buffer to the required concentration. The RNA/pDNA was dissolved in 100 mM sodium acetate buffer to reach the desired concentration ~168 μg/mL. The concentration of NAT is determined by Nanodrop (Thermo Scientific™). This information was used to establish the desired concentration of NAT to mix with the Lipid Mix in ethanol in a NanoAssemblr® Ignite™ instrument as described in Example 2.

### Example 2

This Example demonstrates the preparation of Lipid Nanoparticles (LNP) via microfluidic mixing used in the Examples of the present application. The NAT was prepared as shown in Example 1. Components of the lipid mixes include ionizable lipid, structural lipid or helper lipid, sterol and stabilizing agent in different molar ratios. Stabilizing agent means any agent including PEG-DMG or as defined in the description supra under that category to stabilize the LNP formation. Lipid mix compositions were prepared in ethanol by combining prescribed amounts of lipids (12.5, 25, or 37.5 mM as required; a mixture of ionizable lipid, structural lipid, sterol and stabilizing agent as described in Table 2) from individual lipid stocks in ethanol. LNPs were then prepared by running the lipid mix composition and NAT through the NanoAssemblr® Ignite™ microfluidic mixer.

The mixing of nucleic acid therapeutics (NAT) and lipids occurred as follows. Ionizable lipids, helper lipids, sterol, and stabilizing agent were mixed at a molar ratio of either 47.5:12.5:38.5:1.5 (VA or VB) or 40:20:37.5:2.5 (CT10) in 100% ethanol. The aqueous phase was prepared by diluting nucleic acid therapeutics (NAT) such as mRNA/saRNA/pDNA solutions in 100 mM sodium acetate buffer (pH 4). The solutions were combined using the NanoAssemblr® Ignite™ with an Ignite™ NxGen™ (DVBM) cartridge (Precision Nanosystems) at a flow ratio of 1:3 (organic phase:aqueous phase) at N/P ratio of 8 or 10, unless otherwise noted, with a total flow rate of 12 mL/min. The resulting LNPs were diluted 25-40 times in 1×PBS (pH 7.4) and the mixture was subjected to downstream processing. Downstream processing included ethanol removal through dialysis in PBS (pH 7), or using Amicon™ centrifugal filters (Millipore, USA) at 2500 RPM, or using tangential flow filtration systems. Particles were concentrated to required target dose.

TABLE 2
Exemplary Lipid Mix Compositions as Defined by Ionizable Lipid/Structural
Lipid/Sterol/Stabilizing Agent Ratio. Stabilizing Agent may be replaced by PEG-DMG

40 mol% IL/40 mol% DOPE/17.5 mol% Chol/2.5 mol% Polyoxyethylene (40) stearate
40 mol% IL/30 mol% DOPE/17 mol% Chol/10 mol% Triolein/2.5 mol% Stabilizer
40 mol% IL/20 mol% DOPE/17.5 mol% Chol/20 mol% Triolein/2.5 mol% Stabilizer
40 mol% IL/40 mol% DOPE/0 mol% Chol/17.4 mol% Triolein/ 2.5 mol% Stabilizer
40 mol% IL/20 mol% DOPE/37.5 mol% Chol/2.5 mol% Tridecyl -D-maltoside
40 mol% IL/20 mol% DOPE/37.5 mol% Chol/2.5 mol% Tween 20
40 mol% IL/20 mol% DOPE/37.5 mol% Chol/2.5 mol% Polysorbate 80
40 mol% IL/20 mol% DSPC/39.5 mol% Chol/0.5 mol% TPGS1000
40 mol% IL/20 mol% DSPC/39.5 mol% Chol/0.5 mol% BRIJTMS10
40 mol% IL/20 mol% DSPC/38.5 mol% Chol/1.5 mol% Polysorbate 80
40 mol% IL/20 mol% DSPC/39.5 mol% Chol/0.5 mol% TPGS 1000
40 mol% IL/20mol% DOPE/37.5 mol% Chol/2.5 mol% Polyoxyethylene (40) stearate)
40 mol% IL/20mol% DOPE/39 mol% Chol/1 mol% Polyoxyethylene (40) stearate)/
40 mol% IL/40mol% DOPE/17.5 mol% Chol/2.5mol% Polyoxyethylene (40) stearate)/
40 mol% IL/20 mol% DOPE & 20 mol% POPC/17.5 mol% Chol/2.5mol%
Polyoxyethylene (40) stearate)
40 mol% IL/40mol% DOPE/17.5 mol% Chol/2.5mol% Polyoxyethylene (40) stearate
47.5 mol% IL/12.5 mol% DOPE/38.5 mol% Chol/1.5 mol% PEG-DMG
47.5 mol% IL/12.5 mol% DSPC/38.5 mol% Chol/1.5 mol% PEG-DMG
47.5 mol% IL/12.5 mol% DPPC/38.5 mol% Chol/1.5mol% PEG-DMG
47.5 mol% IL/12.5 mol% SOPC/38.5 mol% Chol/1.5 mol% PEG-DMG
50 mol% IL/10 mol% DOPC/38.5 mol% Chol/1.5 mol% PEG-DMG 2000
50 mol% IL/10mol% DSPC/37.4 mol% Chol/2.5 mol% Polyoxyethylene (40) stearate
50 mol% IL/10 mol% DSPC/38.5 mol% Chol/1.5 mol% PEG-DMG
50 mol% IL/10 mol% DSPC/37.5 mol% Chol/2.5 mol% BRIJTM S20
50 mol% IL/10 mol% DSPC/40 mol% Chol/2.5 mol% TPGS1000
50 mol% IL/10 mol% DSPC/37.5 mol% Chol/2.5 mol% BRIJTMS10
50 mol% IL/10 mol% DSPC/38.5 mol% Chol/1.5 mol% BRIJTMS10
50 mol% IL/10 mol% DSPC/37.5 mol% Chol/2.5 mol% Polyoxyethylene (4) lauryl ether
50 mol% IL/10 mol% DSPC/38.5 mol% Chol/1.5 mol% Polysorbate 80
50 mol% IL/10 mol% DSPC /37.5 mol% Chol/2.5 mol% Polysorbate 80
50 mol% IL/10 mol% DSPC/39.5 mol% Chol/0.5 mol% Polysorbate 80
50 mol% C12-200/10 mol% DSPC/38.5 mol% Chol/1.5 mol% PEG-DMG
50 mol% IL/10mol% DOPC/37.5 mol% Chol/2.5 mol% Polyoxyethylene (40) stearate)
50 mol% IL/10mol% DOPE/37.5 mol% Chol/2.5 mol% Polyoxyethylene (40) stearate)
50 mol% IL/20mol% DOPE/27.5 mol% Chol/2.5 mol% Polyoxyethylene (40) stearate)
50 mol% IL/30mol% DOPE/19 mol% Chol/1 mol% Polyoxyethylene (40) stearate)
50 mol% IL/10 mol% DSPE/38.5 mol% Chol/1.5 mol% PEG-DMG 2000
50 mol% IL/10 mol% DSPC/38.5 mol% Chol/1.5 mol% PEG-DMG 2000

### Example 3

This Example describes the methods used in the following Examples for measuring the size, polydispersity index (PDI) and encapsulation efficiency (EE) of LNPs.

Size and PDI of the LNPs was measured by Dynamic Light Scattering (DLS) using a ZetaSizer™ Nano ZS™ (Malvern Instruments). He/Ne laser of 633 nm wavelength was used as the light source. Data were measured from the scattered intensity data conducted in backscattering detection mode (measurement angle=) 173°. 0.5 to 2 μL of the sample was placed in a cuvette and diluted with PBS (0.3 mL). Measurements were an average of 10 runs of two cycles each per sample. Z-Average size was reported as the particle size and is defined as the harmonic intensity averaged particle diameter. EE of the LNPs was measured by Quant-iT™ RiboGreen® RNA reagent. These LNP characteristics, as well as the results of the nucleic acid EE for the LNP in the various lyophilization buffers (LB) are described in the following examples.

### Example 4

This Example describes the lyophilization process and how lyophilization buffers were evaluated in the following Examples.

Lyophilization buffers (lyo buffer) were prepared in a Base Composition (BC, 10% sucrose in 20 mM Tris and 1×PBS). The weight/volume percentage of each lyo reagent in base composition is described in Table 3. Once the LNPs reached desired target concentrations following manufacturing (40, 80, and 120 μg/mL) as described in Example 2, they were mixed with lyophilization buffer in the ratio of 1:1, 1:2 and 1:4 (V:V). The final solution volume, 200 to 300 μL, was transferred to UPLC 2-mL glass vials or Afton's Ready-To-Fill® sterile vials (2 mL). Once the required concentration was achieved, the lyo buffer mixed LNPs were filter sterilized using 0.2 μm filters in aseptic conditions.

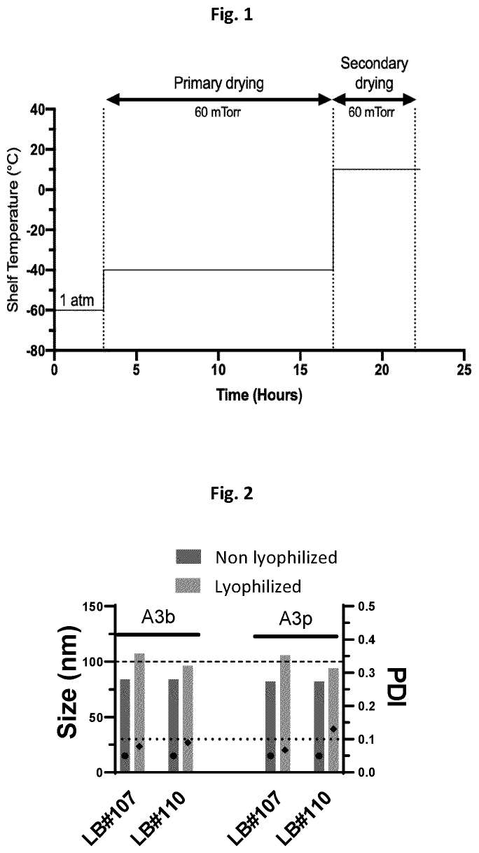

The lyo buffer mixed LNPs were lyophilized using a lyophilizer (freeze dryer) instrument (SP Scientific Model #ADP-S2XL-EOA-X; Serial #326328) by freezing at −60° C. for 3 h, then first desiccation at −40° C./0 min/840 min/60 mTorr, and then followed by a second desiccation at 10° C./0 min/320 min/60 mTorr. A representative schematic of lyophilization process workflow is shown in FIG. 1, which details the different drying cycles. Upon completion of secondary drying, the obtained lyophilized cake or lyo-cakes were stored at different temperatures (RT, 4° C., or −20° C.) for 24 h, one week, three months, or six months (below examples). After the desired storage time, the lyo cakes were reconstituted in 1×PBS to the target NAT concentration/volume (1:1, V/V) prior to any cell or animal treatments as described in the following Examples unless otherwise mentioned. After reconstitution, final encapsulation efficiency was measured by Quant-iT™ RiboGreen® RNA reagent and kit (Invitrogen) following manufacturer directions and the size of the LNPs was measured by Dynamic Light Scattering (DLS) using a ZetaSizer™ Nano ZS™ (Malvern Instruments).

To evaluate the efficiency of lyo buffers, LNP formulations were prepared with different mRNAs (eGFP mRNA, hEPO mRNA, or FLuc mRNA), saRNAs (A3 or A5) or pDNA in either 47.5 mol % IL/12.5 mol % DOPE/38.5 mol % Chol/1.5 mol % PEG-DMG (VA), 47.5 mol % IL/12.5 mol % DSPC/38.5 mol % Chol/1.5 mol % PEG-DMG (VB), or 40 mol % Ionisable lipid/20 mol % DSPC/37.5 mol % Cholesterol/2.5 mol % BRIJ™ S10 (CT10) composition, as described in the following Examples. Potency of lyo buffers were evaluated with various ionizable lipids and helper lipids comprising LNPs encoded with either hEPO mRNA or FLuc mRNA as described in the following Examples. Lyophilization compositions were compared with the base composition (“BC”, 10% sucrose in 20 mM Tris and 1×PBS in solution form or when reconstituted) either as lyophilized LNP cake or as a frozen suspension wherever possible (BC, −80° C.), or to a fresh LNP in PBS (not lyophilized).

TABLE 3
Lyophilization Buffer (LB) Compositions

| | Lyophilization buffer composition in a base of 20 mM Tris/ | % of Lyo Reagent |
|---|---|---|
| LB # | 10% sucrose unless otherwise indicated (such as for LB #9,189) | (Weight/Volume) |
| BC (base | Sucrose 10%, Tris 20 mM | 0 |
| composition) | | |
| 3 | Sucrose (25%) | 0.25 |
| 9 | Sucrose, Trehalose, polyvinylpyrrolidone 10 kDa | 15:10:1.25 |
| 31 | poly(vinyl alcohol) (9000 Da) | 0.5 |
| 54 | sorbitol: poly(vinyl alcohol) (9000 Da) | 9.5:0.5 |
| 82 | poly(ethylene glycol) (3000 Da) | 1 |
| 101 | Propylene glycol | 3.75 |
| 107 | polyvinylpyrrolidone (1000 Da) | 1.25 |
| 108 | poly(N-vinyl caprolactam) (1300 Da) | 1.25 |
| 109 | Sarcosine | 10 |
| 110 | 2-hydroxylpropyl-beta-cyclodextrin (1400 Da) | 2.50 |
| 112 | L-arginine | 2.5 |
| 113 | L-arginine | 5 |
| 116 | polyvinyl alcohol (9000 Da) | 0.25 |
| 119 | 2-hydroxylpropyl-beta-cyclodextrin (1400 Da) | 5 |
| 120 | Glycerol | 10 |
| 134 | Trehalose | 15 |
| 135 | Trehalose: PEG (400 Da) | 10:1 |
| 136 | Trehalose: PEG (400 Da) | 4:1 |
| 140 | Trehalose: Poloxamer 407 (Pluronic F 127) | 200:1 |
| 142 | Polypropylene glycol (400 Da) | 0.50 |
| 143 | Polypropylene glycol (400 Da) | 1.25 |
| 144 | polypropylene glycol (400 Da) | 2.50 |
| 147 | Dextran (1.5 kDa) | 1.25 |
| 148 | Dextran (1.5 kDa) | 2.50 |
| 157 | Triglycerol | 10 |
| 161 | Poly(acrylamide) (24.5 kDa) | 1.25 |
| 162 | Poly(N,N-dimethyl acrylamide) (31 kDa) | 1.25 |
| 163 | poly(N,N-diethyl acrylamide) (19.5 kDa) | 1.25 |
| 164 | poly(2-ethyl-2-oxazoline) (60 kDa) | 1.25 |
| 166 | Beta-cyclodextrin Polymer (2 kDa) | 1.25 |
| 167 | amine terminated 4-arm PEG (2 kDa) | 1.25 |
| 168 | glycerol ethoxylate (1 kDa) | 1.25 |
| 170 | betadex sulfobutyl ether sodium (1451 Da) | 1.25 |
| 171 | Propylene glycol | 15 |
| 178 | 6-arm branched PEG (5.2 kDa) | 1.25 |
| 179 | 5-arm branched PEG (6 kDa) | 1.25 |
| 180 | 3-arm branched PEG (11.5 kDa) | 1.25 |
| 181 | carboxy terminated PEG with sorbitol core (10 kDa) | 1.25 |
| 182 | 6-arm branched PEG (5.2 kDa) | 2.50 |
| 186 | polyvinylpyrrolidone (1.0 kDa) | 2.50 |
| 187 | n-Octanoylsucrose | 0.50 |
| 188 | Trimethyl propane ethoxylate | 2.50 |
| 189 | 2-hydroxylpropyl-beta-cyclodextrin (1.4 kDa)** | 2.50 |
| 192 | N, N-dimethyl acrylamide (31 kDa) | 1.25 |
| 193 | polyacrylamide (24.5 kDa) | 1.25 |
| 208 | Polypropylene glycol (400 Da) | 0.25 |
| 218 | poly(N-vinyl caprolactam) (1.3 kDa) | 0.5 |
| 241 | poly(N-vinyl caprolactam) (1.8 kDa) | 1.25 |
| 260 | poly(N-vinyl caprolactam) (1.8 kDa) | 2.5 |

**10 mM Tris buffer was used instead of 20 mM.

### Example 5

To find a successful lyophilization protocol for mRNA vaccines, various lyo-buffer compositions were evaluated for delivery of saRNAs encoding vaccine antigen. saRNAs encoding for full length spike protein was used. The physico-chemical properties such as size, polydispersity (PDI), and encapsulation efficiency (EE) were measured for lyophilized LNPs (post-lyo) and compared with BC controls (LNPs lyophilized with BC) and fresh LNPs (no lyophilization).

LNPs comprising PNI 516 as ionizable lipid encapsulating saRNAs encoding spike antigen (SARS-Cov-2 spike protein, either A3b, A3p or A5) were manufactured at target concentration of 40, 80, and/or 120 μg/mL were diluted at 1:1, 1:2 and 1:4 LNP to lyo buffer (v:v) in a solution volume of 200 μL in a UPLC 2-mL glass vial. LNP formulation compositions and lyo buffer composition are described below. The LNP were lyophilized with different lyo buffers added to the base composition of 10% sucrose and 20 mM Tris. Desiccation was performed by freezing at −60° C. for 3 h, then desiccation at −40° C./0 min/840 min/60 mTorr, and then a second desiccation at 10° C./0 min/320 min/60 mTorr. The lyophilized cakes were stored at RT for 24 hr. An additional test was performed for lyo cakes stored at −20° C., 4° C., and RT for one week for LNPs comprising A3 saRNAs. Reconstitution was done in 200 μL PBS.

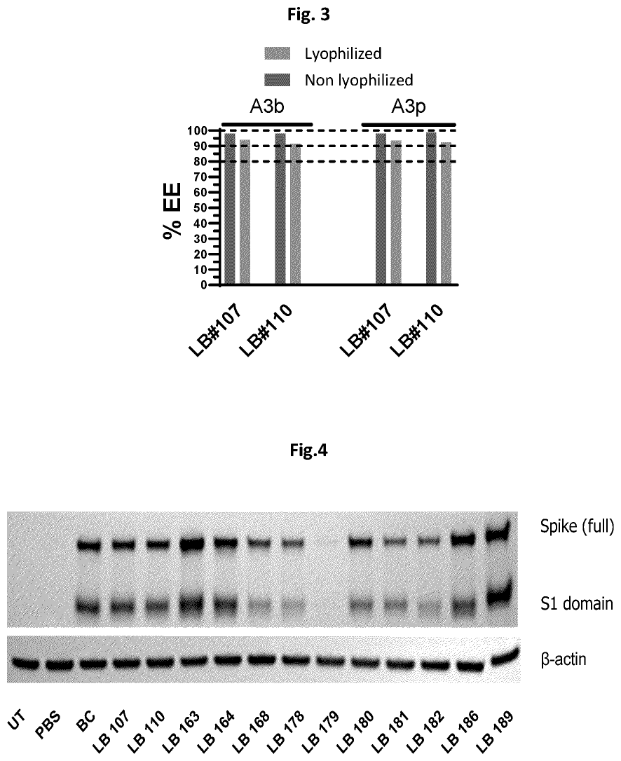

After reconstitution in 1×PBS, size, PDI, and encapsulation efficiency were measured. LB #107, 163, 186, 189, and 192 showed good performance in protecting the size and EE of LNPs of PNI 516 encapsulated with A3 saRNA in a therapeutically viable range (Table 4). LB #107, 110, 164, 168, 178, 179, 181, 182, 186, and 189 showed good performance in protecting the size and EE of LNPs with PNI 516 and A5 saRNAs, where the lyo-cakes were stored at RT for 24 hours (Table 5). FIGS. 2 and 3 show the size, PDI, and EE for A3 saRNA LNPs using LB #107 and LB #110. Both lyo-buffers (LB #107 and #110) protect the size and EE of LNPs in a therapeutically relevant range. LB #110 was found to have least change in size irrespective of the payloads tested. LB #189 retained its minimal PDI as shown in Table 4.

With some of the LBs in Table 1, such as Sucrose alone, they were not pursued further for because of limitations in the early results. For example, LB #3 (Sucrose, 25%), was highly viscous. Additionally, based on the data we achieved, no improvements were found in terms of CQAs and potency when sucrose % increased from 10% to 25% in the absence of additional lyophilization reagents. Sucrose 10% in Tris 20 mM buffer was established as the Base Composition (BC) for the addition of lyophilization agents, and was used as the control in experiments.

TABLE 4
Physiochemical properties of lyophilized, reconstituted spike protein
encoded A3 saRNA LNPs, using ionizable lipid 516 and lipid
mix 47.5 mol % IL/12.5 mol % DSPC/38.5 mol %
Chol/1.5 mol % PEG-DMG.

| | storage | | | Av. PDI - lower is | | Av. EE | |
|---|---|---|---|---|---|---|---|
| | temp | Av. Size (nm) | | better | | | post- |
| LB# | (° C.) | control | post-lyo | control | post-lyo | control | lyo |
| BC | −80 | 65.2 | 68.1 | 0.06 | 0.08 | 99.1 | 91.6 |
| | −20 | 65.2 | 82.1 | 0.06 | 0.19 | 99.1 | 92.9 |
| | 4 | 65.2 | 66.6 | 0.06 | 0.04 | 99.1 | 91.6 |
| | RT | 65.3 | 111.2 | 0.06 | 0.15 | 99.3 | 92.3 |
| 107 | −20 | 65.2 | 99.4 | 0.06 | 0.22 | 99.1 | 97.9 |
| | 4 | 65.2 | 65.6 | 0.06 | 0.05 | 99.1 | 97.9 |
| 110 | −20 | 65.2 | 139.5 | 0.06 | 0.06 | 99.1 | 97.5 |
| | 4 | 65.2 | 135.4 | 0.06 | 0.03 | 99.1 | 97.2 |
| 163 | −20 | 65.2 | 65.8 | 0.06 | 0.07 | 99.1 | 99.0 |
| | 4 | 65.2 | 63.2 | 0.06 | 0.08 | 99.1 | 98.2 |
| 163 | RT | 65.3 | 120.3 | 0.06 | 0.16 | 99.3 | 59.1 |
| 186 | −20 | 65.2 | 69.9 | 0.06 | 0.09 | 99.1 | 96.4 |
| | 4 | 65.2 | 77.2 | 0.06 | 0.12 | 99.1 | 97.3 |
| | RT | 65.3 | 86.8 | 0.06 | 0.13 | 99.3 | 95.0 |
| 189 | −20 | 65.2 | 84.0 | 0.06 | 0.02 | 99.1 | 97.6 |
| | 4 | 65.2 | 81.0 | 0.06 | 0.02 | 99.1 | 98.4 |
| 192 | −20 | 65.2 | 70.8 | 0.06 | 0.14 | 99.1 | 97.1 |
| | 4 | 65.2 | 66.2 | 0.06 | 0.08 | 99.1 | 97.0 |
| 192 | RT | 65.3 | 135.8 | 0.06 | 0.15 | 99.3 | 95.6 |
| 193 | −20 | 65.2 | 130.3 | 0.06 | 0.27 | 99.1 | 98.0 |
| | 4 | 65.2 | 65.4 | 0.06 | 0.10 | 99.1 | 97.4 |

TABLE 5
Physiochemical properties of LNPs comprising IL PNI 516 and A5
saRNA, using same lipid mix, before and after lyophilization.

| | Av Size | | Av PDI | | EE | |
|---|---|---|---|---|---|---|
| LB # | control | post-lyo | control | post-lyo | control | post-lyo |
| BC | 70.8 | 71.0 | 0.09 | 0.08 | 97.0 | 93.5 |
| 107 | 70.8 | 96.1 | 0.09 | 0.16 | 97.0 | 92.5 |
| 110 | 70.8 | 73.6 | 0.09 | 0.11 | 97.0 | 93.3 |
| 163 | 70.8 | 147.8 | 0.09 | 0.20 | 97.0 | 51.2 |
| 164 | 70.8 | 73.3 | 0.09 | 0.10 | 97.0 | 90.9 |
| 168 | 70.8 | 82.4 | 0.09 | 0.12 | 97.0 | 61.8 |
| 178 | 70.8 | 80.4 | 0.09 | 0.16 | 97.0 | 41.8 |
| 179 | 70.8 | 80.5 | 0.09 | 0.14 | 97.0 | 35.7 |
| 180 | 70.8 | 115.8 | 0.09 | 0.25 | 97.0 | 60.8 |
| 181 | 70.8 | 77.3 | 0.09 | 0.14 | 97.0 | 65.9 |
| 182 | 70.8 | 80.2 | 0.09 | 0.15 | 97.0 | 61.8 |
| 183 | 70.8 | 89.7 | 0.09 | 0.09 | 97.0 | 92.3 |
| 186 | 70.8 | 89.0 | 0.09 | 0.13 | 97.0 | 92.0 |
| 189 | 70.8 | 72.7 | 0.09 | 0.09 | 97.0 | 93.8 |

### Example 6

This Example demonstrates the potency of lyophilized saRNA LNPs using Western Blots. LNPs comprising PNI 516 encoding SARS-COV-2 full length spike protein was formulated with 47.5 mol % IL/12.5 mol % DSPC/38.5 mol % Chol/1.5 mol % PEG-DMG composition according to the regular formulation procedure mentioned in the above examples. Various 10 lyo-buffer compositions were tested to preserve the in vitro potency of saRNA LNPs. Lyophilized cakes were then reconstituted with 1×PBS, sterilized, analyzed (size and EE) and used for in vitro cell treatment. LNPs lyophilized with BC composition (BC) and PBS treatment (no LNPs) were used as controls.

Western blot: HEK-293 cells were seeded for 2 days on a 6-well plate (0.3×10⁶ cells/well) and then treated with lyophilized samples for 1 day at 1 μg/mL. Cell lysates were made using an IP lysis buffer. The protein quantification was performed using a BCA kit according to the supplier (Pierce™ BCA Protein Assay Kit) protocol. The protein samples were denatured using a loading dye and 2-mercapto ethanol. An SDS-PAGE was run in a Mini Gel Tank (175 V, 500 mA, 90 min). The gel was transferred to a nitrocellulose membrane (Thermo Fisher) using an iBlotTM2 device (Thermo Fisher). After blocking the membrane, the membrane was stained with primary antibody for 2 h at RT on a shaker followed by staining with secondary antibody. The blots were developed using a chemiluminescent substrate. The blots were imaged using iBright instrument (Thermo Fisher). The spike protein expression was determined qualitatively and compared to controls to assess the performance of the lyophilized LNPs.

FIG. 4 is a western blot of the cell protein of treated cells.

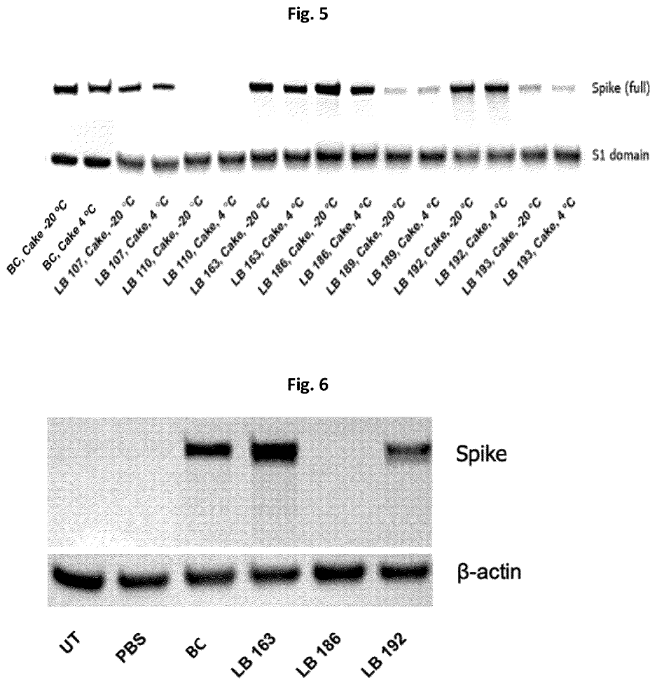

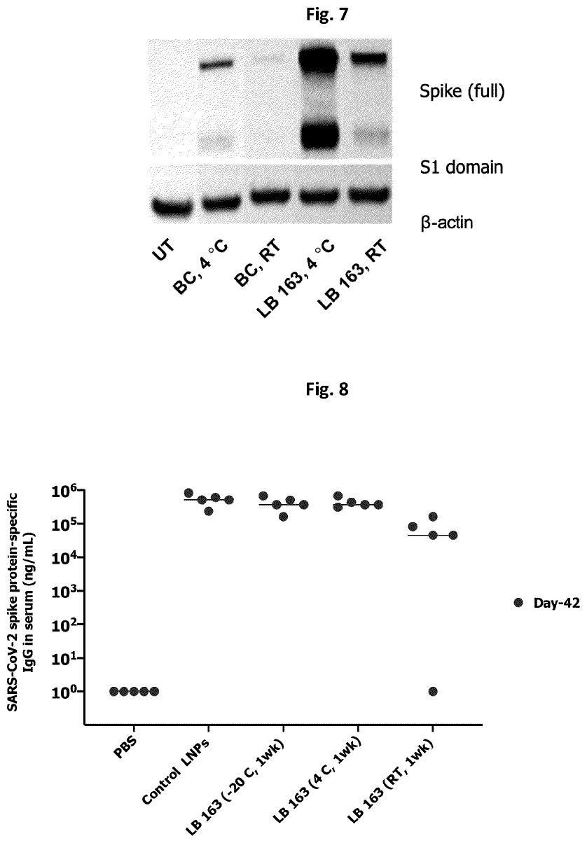

LNP lyophilized with LB #s 107, 110, 163, 164, 180, 186, and 189 showed equal or superior performance compared to BC lyophilized cakes. In this case, the lyo cakes were stored at RT for 24 hr. In another experiment, and the lyophilized A3 saRNA LNP cakes were stored at three different temperatures (−20° C., 4° C., and RT) for one week. As it is shown in FIG. 5 and FIG. 6, LB #163 showed superior performance in expression of spike protein at all three different temperatures. FIGS. 5 and 6 also suggest that LB #107, 186, and 192 preserved the saRNA activity at −20° C. and 4° C. as well. To evaluate the long-term in vitro potency LB #163, A3 saRNA LNP lyo cake comprising LB #163 was stored for three months at 4° C. and RT. LB #163 protected saRNA activity at both temperatures compared to BC, particularly at 4° C. Results are shown in FIG. 7.

### Example 7

This Example demonstrates the ability of the lyophilization buffers to preserve the activity of saRNA-LNP vaccines in mice. Mice were administered (IM) lyophilized LNPs comprising ionizable lipid PNI 516 encoding SARS-COV-2 full length spike protein (A5 saRNA) using VB composition, reconstituted in 1×PBS. Lyophilized vaccines (cakes) were stored for 7 days at −20° C., 4° C., and RT. PBS and cryo-stored LNPs with BC were used as controls. Blood was drawn after Day 42 by tail vein and sera prepared according to standard procedures. Six-week serum analysis for SARS-COV-2 Spike protein specific IgGs was performed using established ELISA procedures.

Nunc Maxisorp™ ELISA plates were coated with 0.5 μg/ml of SARS-COV-2 (2019-nCOV) Spike S1+S2 ECD-His Recombinant Protein (Cat #40589-V08B1, Sino Biologicals, Beijing, China) using PBS overnight. Mouse serum was diluted (1:40000) using 1×ELISA Assay Diluent B (5×) (Cat #421205, BioLegend, San Diego, USA). Standards were prepared using SARS-COV-2 (2019-nCOV) Spike Neutralizing Antibody, Mouse Mab (Cat #40591-MM43, Sino Biologicals) (10-0.16 ng/ml). HRP Goat anti-mouse IgG (minimal x-reactivity) Antibody (Cat #405306, BioLegend) is used for detection. Western TMB substrate is used to develop colour formation, stopped using HCl, and the readings were recorded at 450 nm. FIG. 8 is a SARS-COV-spike protein specific IgG measurement. LB #163 performed equally well compared to cryo-stored LNP controls at all three different storage temperatures (−20° C., 4° C., and RT).

### Example 8

This example demonstrates the ability of the inventive lyophilization buffers to protect mRNA LNPs encoding human EPO protein (hEPO).

Culture conditions of HEK 293 cells for EPO expression: HEK 293 cells were seeded in 6-well plates at 0.3×10⁶ cells/well in 2 mL of complete DMEM (Gibco) and allowed to grow for 48 hours at 37° C. in 5% CO₂. The lyophilized hEPO mRNA-LNPs were stored at desired temperatures for 24 hr, reconstituted in 1×PBS, and added to the cells at 1 μg/mL of mRNA along with controls. After 48 h of incubation, the cell suspensions were harvested and spun at 1200 g for 5 min. The supernatant was analyzed to measure the concentration of hEPO protein using Simple Plex™ Human Erythropoietin Cartridge on the ELLA™ instrument (Protein Simple™ by Biotechne).

Lyophilization: LNPs encoding hEPO mRNA were formulated as mentioned in the above examples. Four lead lyo-buffer compositions were tested to preserve the in vitro potency of mRNA LNPs, which were formulated in VB composition (47.5 mol % PNI 516/12.5 mol % DSPC/38.5 mol % Chol/1.5 mol % PEG-DMG). LNPs lyophilized with BC or PBS treatment (no LNPs) were used as controls. Lyophilized cakes were stored for 24 hr at different temperatures (RT and 4° C.), reconstituted in 1×PBS, sterilized, analyzed (size and EE) and used for cell treatment. The results are shown in FIG. 9. Fresh LNPs had better activity than the BC buffer lyophilized LNPs showcasing that basic composition comprising 20 mM Tris and 10% sucrose is not able to protect the mRNA when LNPs were lyophilized. LNPs lyophilized and reconstituted with lyo buffers (LB #163, #189, #110, and #107) retained its mRNA activity and acting better than BC buffer composition at both temperatures (4° C. and RT). Corresponding size and encapsulation efficiency (%) of the lyophilized LNPs were measured and shown in Table 6.

TABLE 6
Physiochemical properties of lyophilized hEPO mRNA-containing LNPs
comprising IL PNI 516 using lipid mix VB composition compared
to control LNPs.

| | Storage | Size (nm) | | PDI | | EE (%) | |
|---|---|---|---|---|---|---|---|
| | temperature | | Post- | | Post- | | Post- |
| LB# | (° C.) | Control | lyo | Control | lyo | Control | lyo |
| BC | RT = 21 | 67.8 | 108.55 | 0.069 | 0.083 | 98.9 | 83.6 |
| 110 | RT | 67.8 | 93.43 | 0.069 | 0.055 | 98.9 | 96.9 |
| 189 | RT | 67.8 | 99.79 | 0.069 | 0.035 | 98.9 | 92.2 |
| 163 | RT | 67.8 | 134.50 | 0.069 | 0.546 | 98.9 | 81.4 |
| 107 | RT | 67.8 | 86.70 | 0.069 | 0.563 | 98.9 | 95.2 |
| BC | 4 | 67.8 | 89.67 | 0.069 | 0.077 | 98.9 | 90.7 |
| 110 | 4 | 67.8 | 95.99 | 0.069 | 0.040 | 98.9 | 95.9 |
| 189 | 4 | 67.8 | 97.91 | 0.069 | 0.015 | 98.9 | 97.1 |
| 163 | 4 | 67.8 | 93.93 | 0.069 | 0.073 | 98.9 | 85.0 |

### Example 9

This Example demonstrates the ability of the inventive lyophilization buffers to protect mRNA LNPs encoding human EPO protein (hEPO)

In another study done with hEPOmRNA, the encapsulation efficiency (%) and size data of the re-constituted (1×PBS) lyo-cakes of PNI 516 and hEPO LNPs after 7 days of lyo-cake storage at designated temperatures (−20° C., 4° C., or RT) was measured. LNP formation and lyophilization was as described in Example 8. Results are provided in Table 7. Fresh LNPs and lyophilized LNPs in BC at respective temperatures (−20° C., 4° C., or RT) are shown as positive controls. The LB #s 107, 110, 163, 186 and 189 retained the size and EEs of LNPs in a therapeutically range (Table 7).

TABLE 7
Physiochemical properties of lyophilized hEPO mRNA-containing
LNPs comprising IL PNI 516 using lipid mix VB composition after
7 days of storage at different temperatures.

| | Storage | | | PDI | | EE (%) | |
|---|---|---|---|---|---|---|---|
| | temperature | Size (nm) | | | Post- | | Post- |
| LB# | (° C.) | Control | Post-lyo | Control | lyo | Control | lyo |
| BC | −20 | 71.42 | 85.90 | 0.036 | 0.067 | 98.3 | 81.2 |
| BC | 4 | 71.42 | 86.78 | 0.036 | 0.092 | 98.3 | 57.6 |
| BC | RT | 71.42 | 98.99 | 0.036 | 0.138 | 98.3 | 75.6 |
| 107 | −20 | 71.42 | 107.15 | 0.036 | 0.039 | 98.3 | 96.2 |
| 107 | 4 | 71.42 | 108.65 | 0.036 | 0.025 | 98.3 | 96.5 |
| 107 | RT | 71.42 | 133.50 | 0.036 | 0.046 | 98.3 | 96.4 |
| 110 | −20 | 71.42 | 80.26 | 0.036 | 0.041 | 98.3 | 95.7 |
| 110 | 4 | 71.42 | 80.95 | 0.036 | 0.040 | 98.3 | 94.9 |
| 110 | RT | 71.42 | 84.46 | 0.036 | 0.076 | 98.3 | 92.4 |
| 163 | −20 | 71.42 | 99.53 | 0.036 | 0.110 | 98.3 | 79.3 |
| 163 | 4 | 71.42 | 116.20 | 0.036 | 0.070 | 98.3 | 78.5 |
| 163 | RT | 71.42 | 101.00 | 0.036 | 0.092 | 98.3 | 71.0 |
| 186 | −20 | 71.42 | 93.54 | 0.036 | 0.063 | 98.3 | 94.7 |
| 186 | 4 | 71.42 | 90.45 | 0.036 | 0.042 | 98.3 | 94.8 |
| 186 | RT | 71.42 | 90.45 | 0.036 | 0.012 | 98.3 | 95.2 |
| 189 | −20 | 71.42 | 90.39 | 0.036 | 0.019 | 98.3 | 96.2 |
| 189 | 4 | 71.42 | 92.13 | 0.036 | 0.027 | 98.3 | 96.6 |
| 189 | RT | 71.42 | 110.45 | 0.036 | 0.023 | 98.3 | 94.9 |

### Example 10

This Example demonstrates the efficacy of lyo buffers using diverse lipid mix compositions, either by varying the ionizable lipid (IL) or helper lipid (HL). LNPs were prepared using different ionizable lipids and or different helper lipids to evaluate the effectiveness of lyophilization buffers in protecting the mRNA. Lyophilized LNPs were stored at appropriate test conditions and/or time periods, and RNA integrity was tested using an in vitro potency assay in HEK-293 cells.

Lyophilization. A 100 μL of LNPs encapsulating hEPO mRNA at 80 μg/mL were mixed with 100 μL of buffer and lyophilized. Post-lyophilization, the cakes were stored at RT for 7 days. The cakes were re-constituted in 1×PBS and size and encapsulation efficiency (%) were established. LNPs stored at −80° C. using BC was used as a positive control.

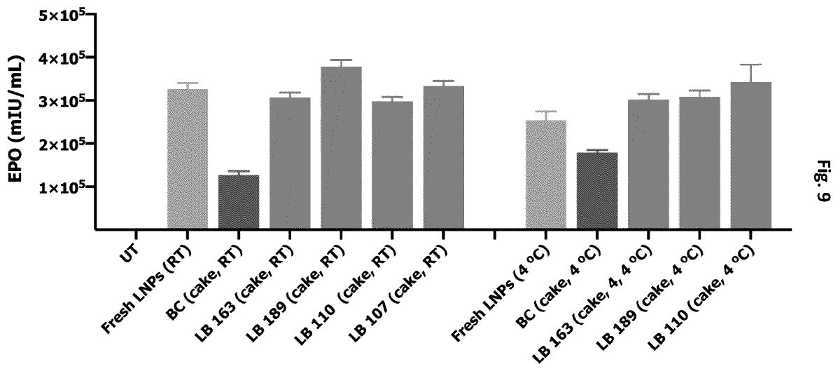

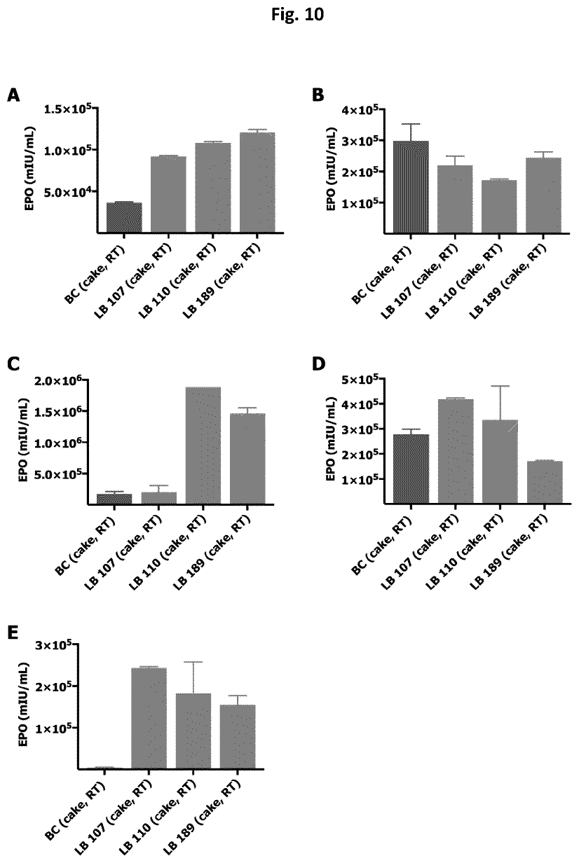

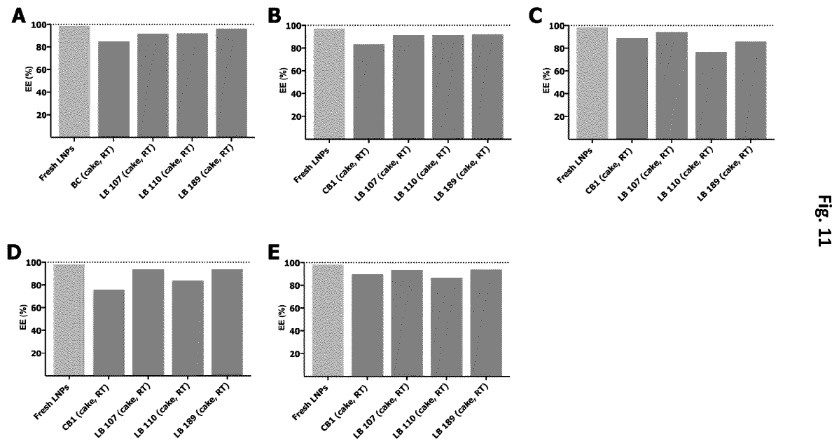

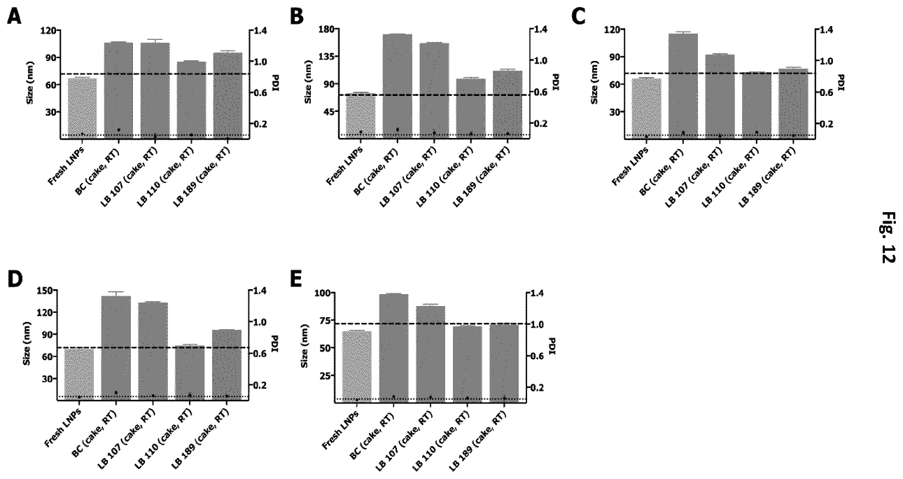

hEPO mRNA-LNPs were encapsulated using varying ionizable lipids PNI 516, PNI 127, MC3, KC2, and BOCHD-C3-DMA in 47.5 mol % IL/12.5 mol % DSPC/38.5 mol % Chol/1.5 mol % PEG-DMG composition along with controls such as Fresh LNPs (no lyophilization) and BC lyophilized LNPs. The lyo cakes were stored for 7 days at RT and reconstituted in 1×PBS. The results are shown in FIG. 10 in which the lyo buffers LB #s 189, 110, and 107 better preserved the expression of EPO than BC and Fresh LNPs. Corresponding size and encapsulation efficiency (%) were measured and shown in FIGS. 11 and 12.

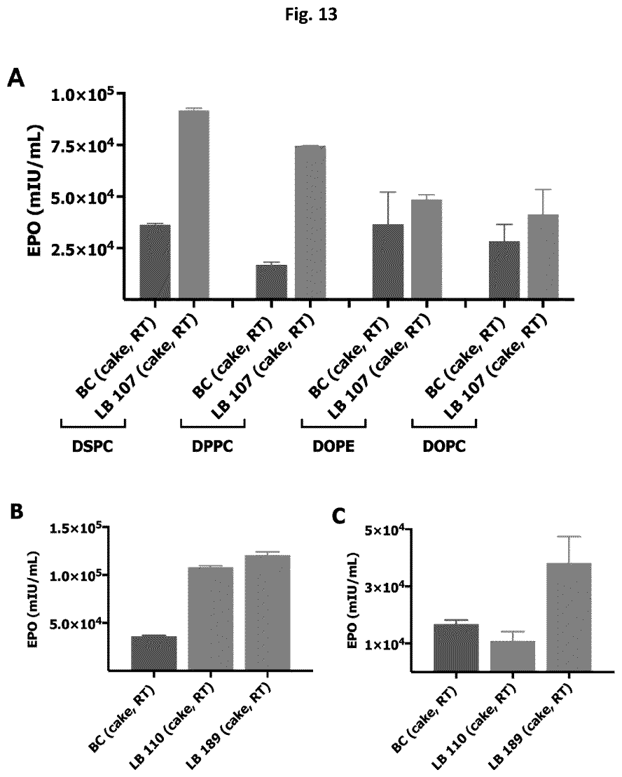

hEPO mRNA-LNPs were prepared by varying helper lipids, DSPC, DOPE, DPPC, or DOPC in the molar ratios of PNI516:Chol:helper lipid:PEG-DMG (47.5:38.5:12.5:1.5), and were lyophilized. The lyophilized cakes were stored at RT for 7 days, re-constituted with 1×PBS, and analyzed for size and encapsulation efficiency (%) (13). Erythropoietin expression in HEK293 cells was measured using Simple Plex™ Human Erythropoietin Cartridge on ELLA™ instrument (FIG. 13). Irrespective of helper lipid used in the LNP composition, LB #107 protected the activity of the LNPs (FIG. 13A for LB #107 for LNP made with DSPC, DPPC, DOPE, DOPC; FIG. 13B for LB #107 and LB #110 for LNP made with DSPC; FIG. 13C for LB #107 and 189 for LNP made with DPPC).

Corresponding encapsulation efficiency (%) of re-constituted cakes after 7 days of cake storage at designated temperatures is shown in Table 8. Fresh LNP (not lyophilized) and base composition (BC) lyophilized cakes at respective temperatures are shown as positive controls.

TABLE 8
Physiochemical properties of lyophilized hEPO mRNA-containing LNPs comprising IL PNI 516
using lipid mix with different helper lipids after 7 days of storage a troom temperature (RT).

| | | Storage | | Size (nm) | | PDI | | EE (%) | |
|---|---|---|---|---|---|---|---|---|---|
| | Helper | temperature | Storage | | Post- | | Post- | | Post- |
| LB# | lipid | (° C.) | time | Control | lyo | Control | lyo | Control | lyo |
| BC | DPPC | RT | 7 days | 56.9 | 103.4 | 0.084 | 0.101 | 98.0 | 90.0 |
| 107 | DPPC | RT | 7 days | 56.9 | 95.6 | 0.084 | 0.063 | 98.0 | 95.6 |
| 110 | DPPC | RT | 7 days | 56.9 | 75.7 | 0.084 | 0.096 | 98.0 | 91.8 |
| 189 | DPPC | RT | 7 days | 56.9 | 77.7 | 0.084 | 0.046 | 98.0 | 96.3 |
| BC | DOPC | RT | 7 days | 66.7 | 158.1 | 0.100 | 0.108 | 97.5 | 90.2 |
| 107 | DOPC | RT | 7 days | 66.7 | 111.5 | 0.100 | 0.070 | 97.5 | 96.1 |
| 110 | DOPC | RT | 7 days | 66.7 | 86.3 | 0.100 | 0.159 | 97.5 | 89.8 |
| 189 | DOPC | RT | 7 days | 66.7 | 131.0 | 0.100 | 0.053 | 97.5 | 94.7 |
| BC | SOPC | RT | 7 days | 60.5 | 175.8 | 0.058 | 0.092 | 98.6 | 88.7 |
| 108 | SOPC | RT | 7 days | 60.5 | 165.2 | 0.058 | 0.031 | 98.6 | 94.3 |
| 110 | SOPC | RT | 7 days | 60.5 | 142.6 | 0.058 | 0.449 | 98.6 | 93.3 |
| 189 | SOPC | RT | 7 days | 60.5 | 130.0 | 0.058 | 0.038 | 98.6 | 96.5 |

### Example 11

This Example demonstrates the efficacy of lyophilization buffers protecting luciferase protein-encoded mRNA-LNPs.

Firefly Luciferase protein expression in HEK cells: HEK 293 cells (ATCC) were seeded in a white 96-well plates at a density of 12×10³ cells/well in 100 μL of complete DMEM (Gibco) and allowed to grow for 24 hours at 37° C. in 5% CO₂. The lyophilized Firefly luciferase protein encoding mRNA LNPs were reconstituted in 1×PBS and added to the cells at doses ranging from 50 ng along with other controls. After 24 hr of treatment, the cells were analyzed for cell viability and luciferase expression was measured using the ONE-Glo™+Tox Assay kit (Promega), following the manufacturer's protocol.

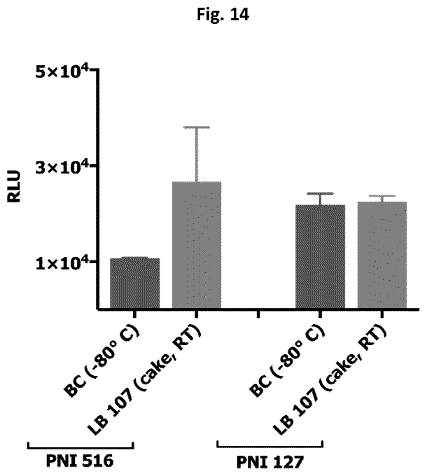

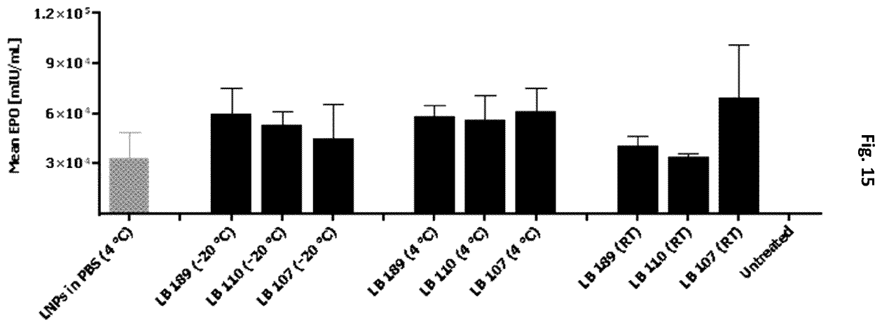

Lyophilization: Fluc mRNA-LNPs were prepared using PNI 516 and 127 in VB composition at N/P-8 and lyophilized by mixing with LB #107. The lyo-cakes were stored at RT for one week and reconstituted in 1×PBS. The control LNPs are BC treated LNPs stored at −80° C. (BC, −80° C.). The results are shown in FIG. 14 in which LB #107 showed similar or better activity in the expression of Luciferase protein in HEK cells with both lipids (PNI 516 and 127) compared to control, BC stored at −80° C. Table 9 shows that LB #107 showed good performance in protecting the size and EE of lyophilized cakes of LNPs of PNI 516 and 127 encoding Luciferase protein mRNA, stored for one week at RT.

TABLE 9
Physiochemical properties of lyophilized luciferase protein-encoded
mRNA-containing LNPs comprising IL PNI 516 using lipid mix 47.5
mol % IL/12.5 mol % DSPC/38.5 mol % Chol/1.5 mol % PEG-DMG
composition compared to control LNPs.

| | | | Size (nm) | | PDI | | EE % | |
|---|---|---|---|---|---|---|---|---|
| | helper | storage | | post- | | post- | | post- |
| LB # | lipid | temp (° C.) | control | lyo | control | lyo | control | lyo |
| BC** | DSPC | −80 ° C. | 79.6 | 85.3 | 0.06 | .05 | 99.9 | 91.5 |
| 107 | DSPC | RT | 79.6 | 91.7 | 0.06 | 0.07 | 99.9 | 95.5 |

**LNPs were preserved in BC (10% sucrose, 10 mMol Tris) at −80 ° C. and measurements were performed after thawing

### Example 12

This Example demonstrates that the inventive lyophilization buffers preserve the activity of mRNA-LNPs in mice: EPO expression in C57BL/6 mice. Mice were administered with reconstituted lyophilized LNPs (cakes were stored for 7 days at −20° C., 4° C., and RT) and fresh LNPs containing EPO encoding mRNA. All the formulations were made using PNI 516 comprising 47.5 mol % IL/12.5 mol % DSPC/38.5 mol % Chol/1.5 mol % PEG-DMG composition. Blood was drawn by tail vein and sera prepared according to standard procedures. 6 hr serum analysis for EPO expression from mice serum was assessed by automated ELISA using ELLA Simple Plex™ Human Erythropoietin Cartridge kit (Protein Simple™ by Biotechne).

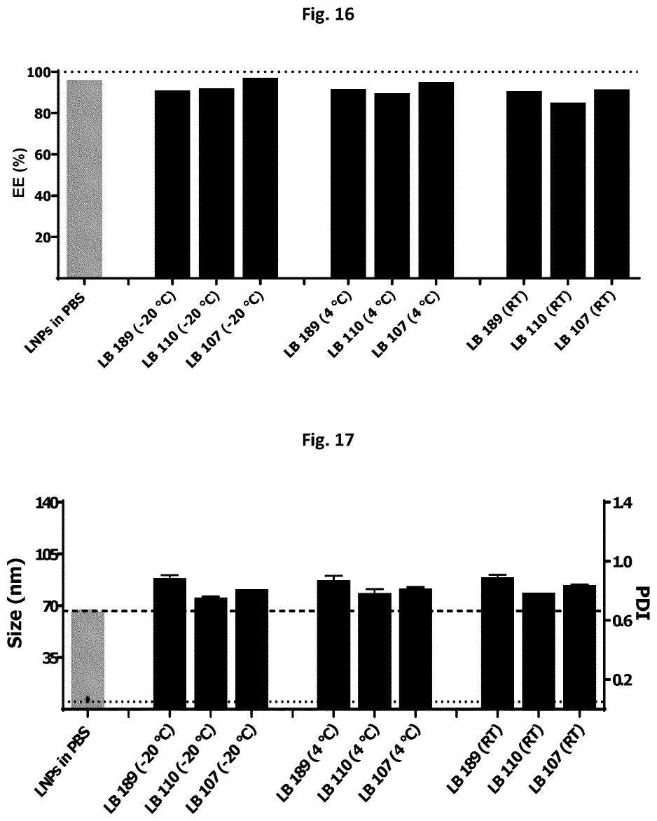

EPO levels in mice are shown in FIG. 15. LB #107 performed best of the tested candidates at RT and 4° C., but LB #189 performed best at minus 20° C. Freshly prepared LNP stored in PBS at 4° C. was used as the control. All reconstituted lyophilized LNPs showed similar or better potency compared to fresh LNP control irrespective of the 7-day aging at various storage temperatures. Encapsulation efficiency is shown in FIG. 16. LB #107, 110, and 189 retained the EE post lyophilization. Size and PDI for these same LNP is shown in FIG. 17. There was no substantial effect on the size and PDI as results of lyophilization as compared to control.

### Example 13

This Example demonstrates that the inventive lyophilization buffers protect plasmid encapsulated LNPs. CMV-eGFP-pDNA plasmid, custom made by GenScript USA Inc, Piscataway, NJ, and PNI 516 lipid were used for LNP formulation. LNPs preparation is as described above. The plasmid was formulated using PNI 516 comprising 47.5 mol % IL/12.5 mol % DSPC/38.5 mol % Chol/1.5 mol % PEG-DMG composition at N/P ratio 8. LNP sample was then lyophilized and the lyo cakes were stored at RT for one week. After reconstitution in 1×PBS, size and EE were measured. As shown in Table 10, LB #s 107, 110, and 189 protected the size and PDI of the LNPs. All the tested LNPs with an average diameter of 93-115 nm, and PDI of 0.2 with encapsulation efficiency greater than 80% before and after lyophilization and reconstitution.

TABLE 10
Physiochemical properties of lyophilized eGFP pDNA-containing
LNPs compared to control LNPs.

| | | storage | size | | PDI | | EE % | |
|---|---|---|---|---|---|---|---|---|
| | helper | temp | | post- | | post- | | post- |
| LB # | lipid | (° C.) | control | lyo | control | lyo | control | lyo |
| PBS | DSPC | RT | 93.1 | | 0.07 | | 86.2 | Not |
| | | | | | | | | detected |
| 107 | DSPC | RT | 93.1 | 115.5 | 0.07 | 0.12 | 86.2 | 84.5 |
| 110 | DSPC | RT | 93.1 | 97.0 | 0.07 | 0.17 | 86.2 | 62.5 |
| 189 | DSPC | RT | 93.1 | 101.1 | 0.07 | 0.10 | 86.2 | 70.2 |

### Example 14

This Example demonstrates that the inventive lyophilization buffers protect eGFPmRNAs. LNPs were prepared as described in Example 2 using PNI 516 and lipid composition VA. The LNPs were lyophilized as described in Example 5 using lyophilization buffers (LB) #s 107, 110, 163, 164, 167, 168, 170, 178, 179, 180, 181, and 189. The lyo-cakes were stored at RT for 24 hours and reconstituted in 1×PBS prior to performing the measurements for the lyophilized LNPs. Each of the lyo buffers protected the size and EE of LNPs of PNI 516 encoding eGFP mRNAs. The lyo buffer LNPs showed similar average size, PDI and EE as fresh LNPs (Table 7b).

TABLE 11
Physiochemical properties of LNPs comprising GFP mRNA using
lipid mix composition VA before and after lyophilization.

| | | Av Size | | Av PDI | | EE | |
|---|---|---|---|---|---|---|---|
| LB # | IL | control | post-lyo | control | post-lyo | control | post-lyo |
| 107 | 516 | 70.0 | 90.0 | 0.07 | 0.09 | 98.0 | 95.1 |
| 110 | 516 | 70.0 | 79.0 | 0.07 | 0.07 | 98.0 | 94.7 |
| 163 | 516 | 70.0 | 87.0 | 0.07 | 0.18 | 98.0 | 93.8 |
| 164 | 516 | 70.0 | 86.0 | 0.07 | 0.07 | 98.0 | 88.4 |
| 167 | 516 | 70.0 | 83.0 | 0.07 | 0.07 | 98.0 | 86.4 |
| 168 | 516 | 70.0 | 89.0 | 0.07 | 0.06 | 98.0 | 84.3 |
| 170 | 516 | 70.0 | 86.0 | 0.07 | 0.11 | 98.0 | 81.6 |
| 178 | 516 | 70.0 | 81.0 | 0.07 | 0.09 | 98.0 | 79.3 |
| 179 | 516 | 70.0 | 87.0 | 0.07 | 0.07 | 98.0 | 87.5 |
| 180 | 516 | 70.0 | 108.0 | 0.07 | 0.02 | 98.0 | 86.1 |
| 181 | 516 | 70.0 | 89.0 | 0.07 | 0.14 | 98.0 | 89.7 |

### Example 15

This Example demonstrates how select lyophilization reagents protect LNPs. LNPs comprising PNI 516 47.5 mol %/12.5 mol % DSPC/38.5 mol % Chol/1.5 mol % PEG-DMG encapsulating mRNA encoding SARS-COV-2 full length spike protein were prepared as above. Lyo buffer #s 101, 109, 112, 113, 119, 120, 134, 135, 136, 140, 142, 143, 147, 148, 157, 161, 162, 166, 171, 182, and 187 (described in Table 3, above) were tested for their ability to preserve the in vitro potency of saRNA LNPs. Lyophilized cakes were then reconstituted with 1×PBS, sterilized, analyzed and used for cell treatment.

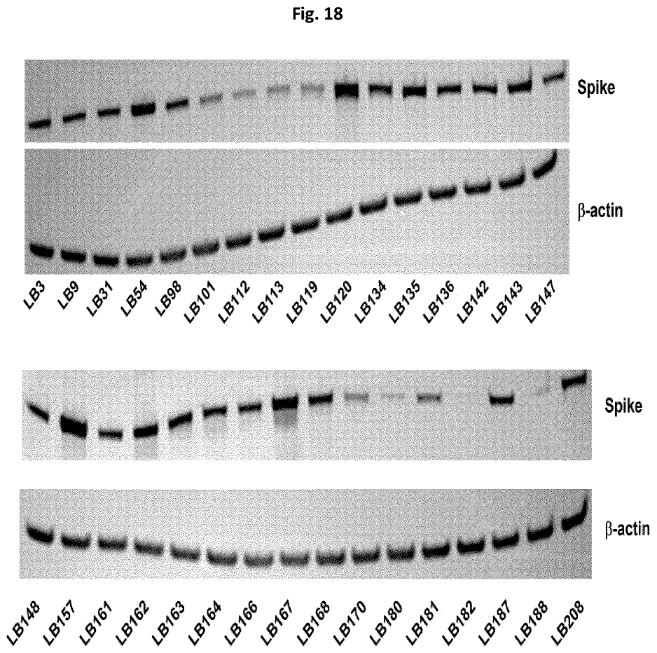

Western blot: HEK-293 cells were seeded for 2 days on a 6-well plate (0.3×10⁶ cells/well) and then treated with lyophilized samples for 1 day at 1 μg/mL. Cell lysates were made using an IP lysis buffer. The protein quantification was performed using a BCA kit according to the supplier (Pierce™ BCA Protein Assay Kit) protocol. The protein samples were denatured using a loading dye and 2-mercapto ethanol. An SDS-PAGE was run in a Mini Gel Tank (175 V, 500 mA, 90 min). The gel was transferred to a nitrocellulose membrane (Thermo Fisher) using an iBlot™M2 device (Thermo Fisher). After blocking the membrane, the membrane was stained with primary antibody for 2 hr at RT on a shaker followed by staining with secondary antibody. The blots were developed using a chemiluminescent substrate. The blots were imaged using iBright instrument (Thermo Fisher). The spike protein expression was determined qualitatively and compared to controls to assess the performance of the lyophilized LNPs. FIG. 18 shows the results of the western blots of protein expression resulting from cells treated with NALNP lyophilized with 31 LB candidates. Protein samples derived from LNP lyophilized with LB #s 54, 120, 134, 135, 148, 157, 167, 168, 187, and 208 showed significant activity post lyophilization.

### Example 16

Table 12. Encapsulation efficiency (EE) of LNPs comprising PNI 516 47.5 mol %/12.5 mol % DSPC/38.5 mol % Chol/1.5 mol % PEG-DMG composition encapsulating A3 saRNA, after 12 days of cake storage at RT. The LNP lyophilized with LB #120, 163, 166 and 167 resulted in a higher EE % compared to LB #54.

TABLE 12
Encapsulation Efficiency After 12 Days

| LB # | EE (%) pos-reconstitution |
|---|---|
| 54 | 56.2 |
| 120 | 78.3 |
| 163 | 77.3 |
| 166 | 83.5 |
| 167 | 60.5 |

### Example 17

In contrast to LB #54, the leads LB #108, LB #167, LB #218 and LB #241 comprising mRNA LNPs showed smaller than 100 nm size, which is a desired quality attribute for parenteral injections. In Table 13, the size of the LNPs comprising IL PNI 516 and hEPO mRNA, using 40 mol % iL/12.5 mol % DSPC/46.0 mol % cholesterol/1.5 mol % PEG-DMG composition after 2 months of cake storage at 4° C. showed much advantage over LB #54, which is PVA.

TABLE 13
hEPO mRNA LNP Size

| LB # | Size (nm) post-reconstitution |
|---|---|
| 54 | 130 |
| 108 | 78 |
| 167 | 82 |
| 218 | 69 |
| 241 | 74 |
| 260 | 73 |

### Example 18

The size changes of LNPs comprising IL PNI 516 and A5 saRNA, using composition as above, after 1 month of cake storage at 4° C. were studied. The leads LB #108, LB #162, LB #164 and LB #167 comprising A5 saRNA LNPs showed less than 100 nm size which is a desired quality attribute for parenteral injections.

TABLE 14
Size of A5 saRNA LNP
after one month storage at 4C

| LB # | Size (nm) post-reconstitution |
|---|---|
| 54 | 150 |
| 108 | 108 |
| 162 | 89 |
| 164 | 94 |
| 167 | 97 |

### Example 19

### In Vivo EPO Expression

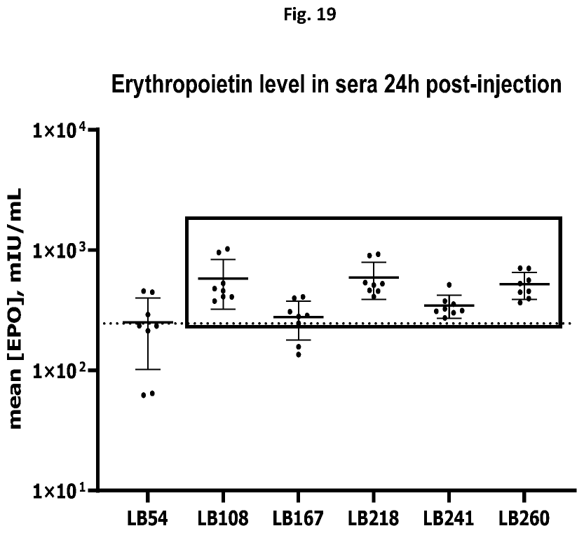

This example describes the procedure used for the hEPO protein expression evaluation of hEPO mRNA-LNPs (reconstituted after 2 months of cake storage at 4° C.) in vivo. LNPs were intramuscularly injected into mice (6-week old male BALB/c mice) at a dose of 0.25 mg/kg (5 μg/20-g mouse). The sera samples were collected 6 h and 24 h post injection. For serum preparation, after collection of the whole blood, the blood was allowed to clot by leaving the collection tube at room temperature for 15-30 minutes. The clot was removed by centrifuging the tubes at 1000-2000×g for 10 min at 4° C. The clear golden-yellow color supernatant was carefully removed and transferred to sterile screw-capped clear polypropylene tube on ice. The serum is then stored in −80° C. until further use. The SARS-COV-2 antigen specific IgG level in sera was determined using enzyme-linked immunoassay (ELISA) assay. The leads LB #108, LB #164, and LB #167 showed better activity than LB #54. The leads LB #108, LB #167, LB #218, LB #241 and LB #260 retained activity compared to PVA comprising LB #54 (FIG. 19).

### Example 20

### Vaccine Expression in Vivo

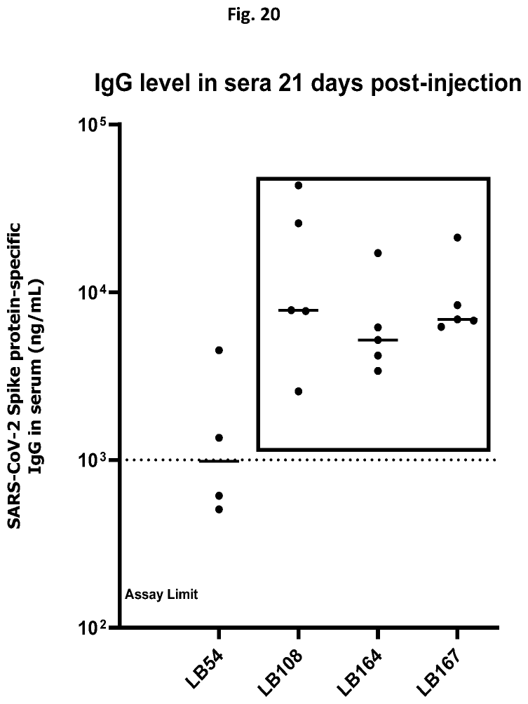

This example describes the procedure used for the SARS-COV-2 expression evaluation In Vivo of SARS-COV-2 expressing A5 PNI saRNA-LNPs reconstituted after 1 month of cake storage at 4° C. LNPs were intramuscularly injected into mice (6-week old male BALB/c mice) at a dose of 0.05 mg/kg (1 μg/20-g mouse) on Day 0. The sera samples were collected 21 days post injection. For serum preparation, after collection, the blood was allowed to clot at RT for 15-30 minutes. The clot was removed by centrifuging the tubes at 1000-2000×g for 10 min at 4° C. The clear golden-yellow color supernatant was carefully removed and transferred to sterile screw-capped clear polypropylene tube on ice. The serum is then stored in −80° C. until further use. The SARS-COV-2 antigen specific IgG level in sera was determined using enzyme-linked immunoassay (ELISA) assay. The leads LB #108, LB #164, and LB #167 showed better activity than LB #54 as shown in FIG. 20.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A lyophilized nucleic acid lipid nanoparticle (NALNP) comprising
(a) a lipid nanoparticle comprising a nucleic acid, and
(b) a lyophilization buffer comprising a sugar, and a lyophilization reagent chosen from a sugar mimicking oligomer/polymer, an amphiphilic thermosresponsive polymer, an ethylene glycol mimicked polymer, a hydrophilic monomer and a hydrophilic polymer.

2. The lyophilization buffer of claim 1, wherein the sugar mimicking oligomer/polymer is n-octanoylsucrose, cyclodextrin, beta-cyclodextrin polymer, dextran, trehalose, carboxy terminated PEG with sorbitol core, Betadex™ sulfobutyl ether sodium, or 2-hydroxylpropyl-beta-cyclodextrin.

3. The lyophilization buffer of claim 1, wherein the amphiphilic thermosresponsive polymer is poly(N-vinyl caprolactam), poly(N, N-dimethyl acrylamide), poly(N,N-diethyl acrylamide), or poly(acrylamide).

4. The lyophilization buffer of claim 1, wherein the ethylene glycol mimicked polymer is 6-arm branched PEG, 5-arm branched PEG, 3-arm branched PEG, trimethyl propane ethoxylate, polyethylene glycol, Poloxamer 407, amine terminated 4-arm PEG, glycerol ethoxylate, or poly(propylene glycol).

5. The lyophilization buffer of claim 1 wherein the hydrophilic monomer or polymer is propylene glycol, glycerol, poly-propylene glycol, triglycerol, poly(vinylpyrrolidone), poly(2-ethyl-2-oxazoline), amino acids, or L-arginine.

6. The NALNP of any one of claims 1-5, wherein the wt/wt ratio of lyophilization reagent to sugar is 1:8.

7. The NALNP of any one of claims 1-5, wherein the wt/wt ratio of lyophilization reagent to sugar is 1:5

8. The NALNP of claim 1, further comprising a pharmaceutically acceptable diluent selected from solutions of Tris, sodium acetate, sodium citrate, dextrose, and saline.

9. The NALNP of claim 1, 6 or 7 wherein the NALNP has a wt/wt ratio of nucleic acid to lyophilization reagent to sugar of 1:125:1000, 1:250:1000, 1:250:2000 or 1:500:2000.

10. The NALNP of claim 1, wherein the sugar is sucrose.

11. The NALNP of any one of claims 1-8, wherein the NALNP is in an anhydrous form.

12. A method for preparing a lyophilized NALNP comprising
(a) mixing a nucleic acid with a lipid mix solution comprising an ionizable lipid, a structural lipid, a sterol, and a stabilizing agent to form a lipid nanoparticle;
(b) combining the lipid nanoparticle obtained in (a) with a lyophilization buffer comprising a sugar and a lyophilization reagent selected from the group consisting of polyvinylpyrrolidone, poly(N-vinyl caprolactam), 2-hydroxylpropyl-beta-cyclodextrin, N, N-dimethyl acrylamide, poly(N,N-diethyl acrylamide), poly(2-ethyl-2-oxazoline), glycerol ethoxylate, amine terminated 4-arm PEG, 6-arm branched PEG, 5-arm branched PEG, and carboxy terminated PEG with sorbitol core, and a pharmaceutically acceptable diluent; and
(c) lyophilizing the combined lipid nanoparticle and lyophilization buffer obtained in (b) to obtain an anhydrous lyophilized NALNP.

13. The method of claim 12, wherein lyophilizing the combined nanoparticle and lyophilization buffer comprises:
(a) freezing the combined lipid nanoparticle and lyophilization buffer at −40 to −90° C. for 60-400 minutes,
(b) drying the combined lipid nanoparticle and lyophilization buffer at −20 to −40° C. and 30-100 mTorr for 700-980 minutes, and
(c) drying the combined lipid nanoparticle and lyophilization buffer at 4-10° C. at 30-100 mTorr for 250-500 minutes.

14. The method of any of claim 12, wherein the lyophilization buffer comprises a pharmaceutically acceptable diluent.

15. The method of claim 14, wherein the pharmaceutically acceptable diluent is Tris, sodium acetate, sodium citrate, dextrose, saline or water.

16. The method of any of claims 10-15, wherein the lyophilization buffer has a wt/wt ratio of lyophilization reagent to sugar to pharmaceutically acceptable diluent between 1:4:40 and 1:8:80.

17. The method of any of claims 10-15, wherein the NALNP has a wt/wt ratio of nucleic acid to lyophilization reagent to sugar of 1:125:1000, 1:250:1000, 1:250:2000 or 1:500:2000.

18. The method of any of claims 10-15, wherein the sugar is sucrose.
